(19) United States
(12) Patent Application Publication
(10) Pub. No.: US 2024/0167102 A1
(43) Pub. Date: May 23, 2024

(54) PREDICTING THERAPEUTIC RESPONSE

(71) Applicant: DERMTECH, INC., La Jolla, CA (US)

(72) Inventors: Michael HOWELL, La Jolla, CA (US); Zuxu YAO, San Diego, CA (US); James ROCK, San Diego, CA (US); Burkhard JANSEN, La Jolla, CA (US)

(21) Appl. No.: 18/548,321

(22) PCT Filed: Mar. 1, 2022
(86) PCT No.: PCT/US2022/018274

§ 371 Date: Aug. 29, 2023

Related U.S. Application Data
(60) Provisional application No. 63/155,665, filed on Mar. 2, 2021.

(51) Int. Cl. C12Q 1/6886; A61K 45/06; C12Q 1/6806; C12Q 1/6809

(52) U.S. Cl. CPC C12Q 1/6886; A61K 45/06; C12Q 1/6806; C12Q 1/6809; C12Q 2600/106; C12Q 2600/158

(57) ABSTRACT

Disclosed herein are methods of assessing response to therapies. Also described herein are methods for using the methods described herein for assessing response to therapies for treating skin diseases or skin conditions.

### CROSS-REFERENCE

This application claims the benefit of U.S. provisional patent application No. 63/155,665 filed on Mar. 2, 2021 which is incorporated herein by reference in its entirety.

### BACKGROUND

Diseases having cutaneous manifestations include some of the most common human illnesses and represent an important global burden in healthcare. Existing methods for assessing optimum treatment of common diseases (such as skin cancer) suffer from invasiveness, high cost, and lengthy clinical trials.

### INCORPORATION BY REFERENCE

All publications, patents, and patent applications mentioned in this specification are herein incorporated by reference to the same extent as if each individual publication, patent, or patent application was specifically and individually indicated to be incorporated by reference in its entirety.

### BRIEF SUMMARY

Current methods for predicting responses to therapeutic intervention rely on evaluating potential therapies using skin biopsies, pathology assessments, and gene signatures within multiple clinical trials (by evaluating clinical response and comparing to sample signatures). These clinical trials can be cost prohibitive and time-consuming which can prevent the true integration of personalized medicine in (dermatological) treatment. By comparing signatures obtained from non-invasive or minimally invasive skin samples with signatures obtained from invasive samples subjected to in vitro treatment exposure models, the clinical outcome in diseases and conditions can be more readily predicted without the use of clinical subjects in clinical trials. The signatures obtained from comparisons provide the capability to predict the outcome of a treatment based on non-invasive sampling without costly clinical trials.

Provided herein are methods for preparing samples from a subject useful for predicting a therapeutic response to a treatment in a subject with a disease or condition having cutaneous manifestations, comprising: a) obtaining a first biological sample and a second biological sample from a single subject; b) identifying a baseline biomarker signature from the first biological sample; c) applying a treatment to the second biological in-vitro for a time period; d) identifying a treatment signature from the second sample after the time period; and e) comparing the baseline signature with the treatment signature to determine an outcome signature to the one or more treatments. Further provided herein are methods wherein the disease or condition is an inflammatory or autoimmune disease. Further provided herein are methods wherein the disease or condition comprises a condition wherein the skin is a target or surrogate target of the cutaneous manifestation. Further provided herein are methods wherein the inflammatory or autoimmune disease is atopic dermatitis, psoriasis, allergy, Crohn's disease, lupus, asthma, or vitiligo. Further provided herein are methods wherein the disease or condition comprises cancer or pre-cancerous conditions. Further provided herein are methods wherein the cancer is melanoma or non-melanoma skin cancers. Further provided herein are methods wherein the melanoma comprises basal cell carcinoma or squamous cell carcinoma. Further provided herein are methods wherein the non-melanoma comprises merkel cell carcinoma or keratinosis. Further provided herein are methods wherein the disease or condition comprises a pre-malignant condition. Further provided herein are methods wherein the pre-malignant condition comprises actinic keratosis. Further provided herein are methods wherein the one or more treatments comprises exposure to radiation. Further provided herein are methods wherein the one or more treatments comprises phototherapy. Further provided herein are methods wherein the radiation comprises ultraviolet, visible, or infrared light. Further provided herein are methods wherein the one or more treatments comprises a therapeutic agent. Further provided herein are methods wherein the therapeutic agent is a topical or systemic agent. Further provided herein are methods wherein the therapeutic agent is a small molecule or peptide. Further provided herein are methods wherein the therapeutic agent comprises an antibody, diabody, scFv, or fragment thereof. Further provided herein are methods wherein the antibody comprises anti-TNF-a, anti-IL17A, anti-IL23p19, anti-IL-4Ralpha, or anti-IL-13. Further provided herein are methods wherein the therapeutic agent comprises a steroid. Further provided herein are methods wherein the therapeutic agent comprises an anti-proliferative. Further provided herein are methods wherein the first sample is non-invasively or minimally invasively sampled. Further provided herein are methods wherein the second sample is non-invasively or minimally invasively sampled. Further provided herein are methods wherein the second sample is invasively sampled. Further provided are methods wherein the first biological sample and the second biological sample are different. Further provided herein are methods wherein the difference between the first biological sample and the second biological sample comprises the sampling method. Further provided herein are methods wherein the difference between the first biological sample and the second biological sample comprises the sampling location on the subject. Further provided herein are methods wherein the difference between the first biological sample and the second biological sample comprises the time the sample was obtained. Further provided herein are methods wherein the first sample is obtained using a method comprising tape stripping, microneedles, and/or blood sampling. Further provided herein are methods wherein the first biological sample is a skin sample. Further provided herein are methods wherein the skin sample comprises the epidermis. Further provided herein are methods wherein the skin sample comprises the stratum corneum. Further provided herein are methods wherein the second biological sample is a skin sample. Further provided herein are methods wherein the skin sample is obtained from a skin biopsy. Further provided herein are methods wherein the method further comprises dividing the second biological sample into a plurality of aliquots. Further provided herein are methods wherein the time period is up to 10 days. Further provided herein are methods wherein the time period is 3-15 days. Further provided herein are methods wherein the first biological sample or the second biological sample is obtained from a lesion. Further provided herein are methods wherein the baseline signature and the treatment signature comprise information about levels of at least one of protein, lipid, mRNA, or miRNA. Further provided herein are methods wherein the baseline signature and the treatment signature comprise information about location and frequency of at least one genetic variant. Further provided herein are methods wherein the baseline signature and the treatment signature comprise information about levels of expression for one or more genes. Further provided herein are methods wherein step e) comprises comparing weighted values of 5 or more genes. Further provided herein are methods wherein comparing comprises comparing weighted values of 1000 or more genes. Further provided herein are methods wherein the baseline signature and the treatment signature comprise the same set of biomarkers. Further provided herein are methods wherein the outcome signature comprises a predictive and/or treatment signature. Further provided herein are methods wherein the method further comprises measuring the set of biomarkers obtained from the second biological sample to generate a treatment signature. Provided herein are methods for preparing a sample useful for differentiating a responder from a non-response to a treatment in a subject with a disease having cutaneous manifestations, comprising: obtaining a test sample from the skin of a subject; identifying a baseline test biomarker signature from the test sample; comparing the baseline test biomarker signature with an outcome signature obtained from any one of the methods described herein; and identifying whether the subject is a responder or non-responder to the treatment based on the comparison. Further provided herein are methods wherein the test sample is obtained using a non-invasive or minimally invasive sampling method. Further provided herein are methods wherein the test sample is obtained using a method comprising tape stripping, microneedles, or blood sampling. Further provided herein are methods wherein the test sample is a skin sample. Further provided herein are methods wherein the skin sample comprises the epidermis. Further provided herein are methods wherein the skin sample comprises the stratum corneum.

Provided herein are methods for preparing a nucleic acid sample from a subject useful for predicting a response to a disease or condition having cutaneous manifestations comprising: extracting nucleic acids and/or proteins from a first biological sample of a subject, wherein the nucleic acids are obtained from the first biological sample; excising a second biological sample from the subject; applying one or more treatments to the second biological sample for a time period, wherein the treatments are applied in-vitro; extracting nucleic acids and/or proteins from the second biological sample; measuring a signature for the first biological sample to generate a baseline signature; measuring a signature for the second biological sample to generate a treatment signature; comparing the baseline signature and the treatment signature to generate an outcome signature corresponding to the one or more treatments. Further provided herein are methods wherein the skin biopsy sample is contacted with keratinocyte basal medium. Further provided herein are methods wherein step a) further comprises detection of nucleic acids corresponding to genes measured in the treatment signature. Further provided herein are methods wherein step a) further comprises detection of proteins and/or lipids measured in the treatment signature. Further provided herein are methods wherein the first biological sample is obtained using a non-invasive or minimally invasive sampling technique. Further provided herein are methods wherein the first biological sample comprises cellular material from the stratum corneum. Further provided herein are methods wherein the stratum corneum has been separated from the remainder of epidermis. Further provided herein are methods wherein the second biological sample comprises cellular material from the epidermis. Further provided herein are methods wherein the second biological sample is obtained from a skin biopsy. Further provided herein are methods wherein comparing comprises correlating the presence or absence of one or more biomarkers from the first biological sample and the second biological sample. Further provided herein are methods wherein comparing comprises correlating the abundance of one or more biomarkers from the first biological sample and the second biological sample.

### BRIEF DESCRIPTION OF THE DRAWINGS

This patent application contains at least one drawing executed in color. Copies of this patent or patent application with color drawing(s) may be provided by the Office upon request and payment of the necessary fee.

FIG. 1A illustrates a schematic diagram describing an exemplary method for assessing a disease or condition having cutaneous manifestations described herein.

FIG. 1B illustrates a schematic diagram describing an exemplary method for assessing an optimum treatment for a disease or condition having cutaneous manifestations described herein.

FIG. 2 illustrates a non-limiting example of a computing device; in this case, a device with one or more processors, memory, storage, and a network interface.

FIG. 3 illustrates a non-limiting example of a web/mobile application provision system; in this case, a system providing browser-based and/or native mobile user interfaces.

FIG. 4 illustrates a non-limiting example of a cloud-based web/mobile application provision system; in this case, a system comprising an elastically load balanced, auto-scaling web server and application server resources as well synchronously replicated databases.

The novel features of the disclosure are set forth with particularity in the appended claims. A better understanding of the features and advantages of the present disclosure may be obtained by reference to the following detailed description that sets forth illustrative embodiments.

### DETAILED DESCRIPTION

Provided herein are methods and systems for predicting therapeutic response to a disease or condition. In some instances, the disease or condition comprises cutaneous manifestations. In some instances, methods and systems comprise obtaining biological samples from a subject, such as a first biological sample and a second biological sample. In some instances, methods and systems comprise obtaining biological samples from a subject, wherein at least one sample is non-invasively or minimally invasively sampled. In some instances, at least one biological sample is then analyzed to obtain a baseline biomarker signature. Signatures described herein in some instances comprise one or more biomarkers. In some instances signatures comprise additional quantitative information related to one or more biomarkers. In some instances at least one biological sample is exposed to a treatment in-vitro, and subsequently analyzed to obtain a treatment signature. By comparing baseline and treatment signatures, and outcome signature in some instances is generated which is predictive of how the subject having a specific baseline signature will respond to the treatment in-vivo. Further described herein are methods of identifying a patient as a responder or non-responder to a specific treatment by comparing the subject's baseline signature (e.g., obtained from a test sample) to a previously determined outcome signature corresponding to a treatment. Further described herein are methods of sample preparation for in-vitro biomarker analysis using non-invasive or minimally invasive techniques. Further described herein are computer-assisted methods and systems for identifying and comparing biomarker signatures.

Provided herein are methods for generating one or more signatures for predicting therapeutic response for the disease on condition having cutaneous manifestations. In some embodiments, the method analyzes at least one biological sample described herein for generating the one or more signatures. In some embodiments, the biological sample is a skin sample. In some embodiments, the biological sample is a liquid biopsy sample. In some embodiments, the biological sample is a blood sample. In some embodiments, the method analyzes a first biological sample obtained from a subject. The first biological sample may be a first skin sample or a first liquid biopsy sample (e.g., a first blood sample). In some embodiments, the first biological sample is obtained from the subject via non-invasive or minimally invasive method. For example, the first skin sample may be obtained by an adhesive tape, a microneedle, or any skin collection method or kit described herein. In some embodiments, the first skin sample is obtained from healthy or normal looking skin. In some embodiments, the first skin sample is obtained from abnormal or lesioned skin. The first skin sample may be used in vitro for measurements and analysis of at least one biomarker isolated from the skin sample used to generate a baseline biomarker signature. In some embodiments, the first biological sample is obtained from an untreated subject. In some embodiments, a second biological sample is obtained from the same subject, where the second biological sample comprising a second skin sample is treated in vitro in the presence of one or more treatments described herein. In some embodiments, at least one biomarker is isolated from the treated second skin sample culture to generate a treatment biomarker signature. In some embodiments, an outcome signature is generated from the comparison of the baseline biomarker signature and the treatment biomarker signature. In some embodiments, the outcome signature predicts therapeutic efficacy or outcome of the one or more treatments for treating the subject's disease or condition. In some embodiments, the outcome signature is used to design a treatment regimen for treating the subject's disease or condition. In some embodiments, the biological sample (e.g., the first or the second skin biological) may be analyzed for a presence or an expression level of at least one biomarker described herein. The biomarker may be either a genetic marker (e.g., genetic mutation or epigenetic marker), a non-genetic marker (e.g., environmental factor), metabolite, lipid, protein, and/or other biomarker described herein. In some embodiments, the at least one biomarker is a protein, lipid, or carbohydrate.

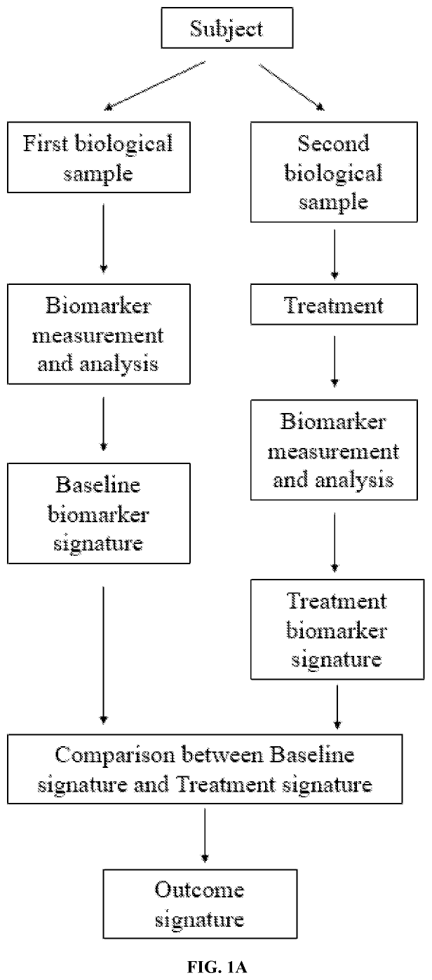

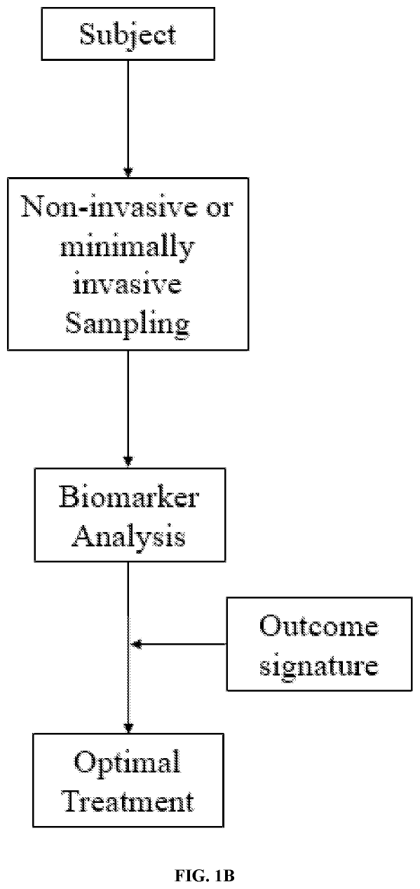

FIG. 1A illustrates an example of generating an outcome signature for treating a subject with a treatment regimen. A first biological sample is obtained from a subject. The first biological in some instances is a skin biopsy sample obtained by the sampling method described herein. The first biological sample in some instances is a liquid biopsy such as blood drawn from the subject. The first sample comprising skin in some instances is used in an in vitro environment. At least one biomarker in some instances is obtained from the first biological sample and analyzed to generate a baseline biomarker signature. A second biological sample may be obtained from the same subject. The second biological sample in some instances is used in vitro and treated with one or more treatments described herein. At least one biomarker may be obtained from the treated second skin sample to generate a treatment biomarker signature. Comparison between the baseline biomarker signature and the treatment biomarker signature generates an outcome signature, which is then used to design a treatment regimen for treating a disease or condition of the subject. The sampling and the analyzing of the biomarkers may be repeated to generate additional rounds of the signatures to alter or titrate the treatment regimen based on the changes of the subject during the course of the treatment. Such titration of the treatment regimen may increase therapeutic response and outcome.

FIG. 1B illustrates an example of applying an outcome signature to a subject suspected of having a disease or condition having cutaneous manifestations. A biological sample in some instances is acquired non-invasively or minimally invasively from the subject, and the biomarker signature from the sample is compared with a previously determined outcome signature for a treatment. Based on the outcome signature, an optimum treatment is selected for the subject.

### Biological Samples

Described herein are methods for obtaining a biological sample. In some instances, biological samples are obtained to identify baseline and treatment biomarker signatures. In some embodiments, the method comprises extracting a nucleic acid, protein, carbohydrate or lipid sample from a biological sample from a subject. In some instances, the biological sample comprises a skin sample. In some instances, the biological sample is obtained using a non-invasive (or minimally invasive) sampling technique. In some instances, the biological sample is obtained from skin or blood. In some embodiments, the non-invasive sampling technique comprises contacting the skin of the subject with an adhesive tape or patch for extracting skin cells. In some instances, the biological sample is obtained from the stratum corneum. In some embodiments, the non-invasive sampling technique comprises contacting the skin of the subject with a microneedle, such as that used for extracting skin cells. In some embodiments, a skin sample is obtained using an invasive or minimally invasive sampling technique. The invasive or minimally invasive sample technique may include using an adhesive tape or patch, where the adhesive tape or patch comprises increased adhesiveness compared to the adhesive tape or patch used for non-invasive sampling. In some embodiments, the invasive or minimally invasive sample technique may include using a microneedle, where the microneedle comprises increased abrasiveness compared to the abrasiveness of a microneedle used for non-invasive sampling. In some embodiments, the biological sample is obtained by swabbing. In some embodiments, the biological sample is obtained by skin biopsy. The skin biopsy may be punch biopsy or shave biopsy. In some embodiments, the skin sample is obtained by hair root sampling (which samples skin that is deeper than the epidermis), buccal smear, or suction blistering. In some instances, the biological sample comprises cells obtained from blood. In some instances, the biological sample comprises skin progenitor cells. In some instances, the biological sample comprises PBMCs. In some instances, the biological sample is further differentiated into skin cells in-vitro. In some instances, a biological sample is contacted with one or more treatments in-vitro. In some instances, a biological sample is cultured in-vitro. Any number of biological samples, in some instances, may be obtained from a subject, such as 1, 2, 3, 4, 5, 6, 7, 8, or more than 9 biological samples.

Biological samples may be prepared for in-vitro use. In some instances, biological samples comprise cells. In some instances, cells are cultured in a media or buffer. In some instances, the medium is keratinocyte basal medium. Cells cultured in some instances do not appreciably grow or divide. In some instances cultured cells are manipulated in such a way to grow or divide. In some instances, cells are maintained in-vitro for at least 1, 2, 3, 4, 5, 6, 7, 8, 9, 10, 11, 12, 13, 14, 15, 16, 17, 18, 19, or at least 20 days. In some instances, cells are utilized in-vitro for about 1, 2, 3, 4, 5, 6, 7, 8, 9, 10, 11, 12, 13, 14, 15, 16, 17, 18, 19, or about 20 days. In some instances, cells are utilized in-vitro for 5-20, 5-15, 8-12, 2-5, 5-10, 10-20, or 15-20 days. In some instances, cells used in-vitro are contacted with one or more treatments. In some instances, biological samples used in-vitro comprise cells obtained from a biopsy. In some instances, biological samples used in-vitro comprise cells obtained from blood.

Biological samples may be obtained from any part of a subject. In some embodiments, a biological sample is obtained from a bodily fluid such as blood, sputum, semen, etc. In some embodiments, a biological sample is obtained from the surface of a subject including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms. In some instances, biological samples are obtained from the same or substantially the same area of a subject. In some instances, biological samples are obtained from a subject at two separate time points. In some instances, the time points are separated by no more than 1 hour, 12 hours, 1 day, 2 days, 15 days, 30 days, 2 months, 6 months, 1 year, 2 years, 5 years, or no more than 10 years.

Biological samples may comprise RNA. In some instances, the nucleic acid comprises RNA (e.g. mRNA). An effective amount of a biological sample contains an amount of cellular material sufficient for performing a diagnostic assay. In some instances, the diagnostic assay is performed using the cellular material isolated from the biological sample. In some embodiments, an effect amount of a biological sample comprises an amount of RNA sufficient to perform a genomic analysis. Sufficient amounts of RNA includes, but not limited to, picogram, nanogram, and microgram quantities. In some embodiments, the RNA includes mRNA. In some embodiments, the RNA includes microRNAs. In some embodiments, the RNA includes mRNA and microRNAs.

In some instances, the nucleic acid is a RNA molecule or a fragmented RNA molecule (RNA fragments). In some instances, the RNA is a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free, circulating RNA.

In some instances, the nucleic acid comprises DNA. DNA includes, but is not limited to, genomic DNA, viral DNA, mitochondrial DNA, plasmid DNA, amplified DNA, circular DNA, circulating DNA, cell-free DNA, complementary DNA (cDNA) or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free, circulating DNA.

A biological sample may be obtained using an adhesive tape or patch from the sample collection kit described herein. In some embodiments, the adhesive tape or patch from the sample collection kit described herein comprises a first collection area comprising an adhesive matrix and a second area extending from the periphery of the first collection area. The adhesive matrix is located on a skin facing surface of the first collection area. The second area functions as a tab, suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use.

In some embodiments, the first collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent. In some embodiments, the adhesive matrix is configured to adhere cells from the stratum corneum of a skin sample.

In some embodiments, the matrix material is sufficiently sticky to adhere to a skin sample. In some embodiments, the matrix material does not cause scarring or bleeding when removed or is not difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant.

In some embodiments, the adhesive patch comprises a flexible material, enabling the patch to conform to the shape of the skin surface upon application. In some instances, at least the first collection area is flexible. In some instances, the tab is plastic. In an illustrative example, the adhesive patch does not contain latex, silicone, or both. In some embodiments, at least some portion of the adhesive patch is made of a transparent material, so that the skin sampling area of the subject is visible after application of the adhesive patch to the skin surface. In some other embodiments, all of the adhesive patch is made of transparent material. The transparency ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical. In some embodiments, the first collection area is circular.

In further embodiments, the adhesive patch of this invention is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector.

In some instances, nucleic acids are stable on the adhesive patch or patches when stored for a period of time or at a particular temperature. In some instances, the period of time is at least or about 1 day, 2 days, 3 days, 4 days, 5 days, 6 days, 7 days, 2 weeks, 3 weeks, 4 weeks, more than 4 weeks, or any period of time in between. In some instances, the period of time is about 7 days. In some instances, the period of time is about 10 days. In some instances, the temperature is at least or about −80° C., −70° C., −60° C., −50° C., −40° C., −20° C., −10° C., −4° C., 0° C., 5° C., 15° C., 18° C., 20° C., 25° C., 30° C., 35° C., 40° C., 45° C., 50° C., or more than 50° C. In some instances, the temperature is no more than −80° C., −70° C., −60° C., −50° C., −40° C., −20° C., −10° C., −4° C., 0° C., 5° C., 15° C., 18° C., 20° C., 25° C., 30° C., 35° C., 40° C., 45° C., 50° C., or no more than 50° C. The nucleic acids on the adhesive patch or patches, in some embodiments, are stored for any period of time described herein and any particular temperature described herein. For example, the nucleic acids on the adhesive patch or patches are stored for at least or about 7 days at about 25° C., 7 days at about 30° C., 7 days at about 40° C., 7 days at about 50° C., 7 days at about 60° C., or 7 days at about 70° C. In some instances, the nucleic acids on the adhesive patch or patches are stored for at least or about 10 days at about −80° C.

The peelable release sheet, in certain embodiments, is configured to hold a single adhesive patch, e.g., 1, or a plurality of adhesive patches, including, but not limited to, 12, 11, 10, 9, 8, 7, 6, 5, 4, 3, 2, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the peelable release sheet is configured to hold about 12 adhesive patches. In some instances, the peelable release sheet is configured to hold about 11 adhesive patches. In some instances, the peelable release sheet is configured to hold about 10 adhesive patches. In some instances, the peelable release sheet is configured to hold about 9 adhesive patches. In some instances, the peelable release sheet is configured to hold about 8 adhesive patches. In some instances, the peelable release sheet is configured to hold about 7 adhesive patches. In some instances, the peelable release sheet is configured to hold about 6 adhesive patches. In some instances, the peelable release sheet is configured to hold about 5 adhesive patches. In some instances, the peelable release sheet is configured to hold about 4 adhesive patches. In some instances, the peelable release sheet is configured to hold about 3 adhesive patches. In some instances, the peelable release sheet is configured to hold about 2 adhesive patches. In some instances, the peelable release sheet is configured to hold about 1 adhesive patch.

Provided herein, in certain embodiments, are methods and compositions for obtaining a sample using an adhesive patch, wherein the adhesive patch is applied to the skin and removed from the skin. After removing the applied adhesive patch from the skin surface, the patch stripping method, in some instances, further comprises storing the applied patch on a placement area sheet, where the patch remains until the skin sample is isolated or otherwise utilized. In some instances, the applied patch is configured to be stored on the placement area sheet for at least 1 week at temperatures between −80° C. and 30° C. In some embodiments, the applied patch is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some instances, the placement area sheet comprises a removable liner, provided that prior to storing the applied patch on the placement area sheet, the removable liner is removed. In some instances, the placement area sheet is configured to hold a single adhesive patch, e.g., 1, or a plurality of adhesive patches, including, but not limited to, 12, 11, 10, 9, 8, 7, 6, 5, 4, 3, 2, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the placement area sheet is configured to hold about 12 adhesive patches. In some instances, the placement area sheet is configured to hold about 11 adhesive patches. In some instances, the placement area sheet is configured to hold about 10 adhesive patches. In some instances, the placement area sheet is configured to hold about 9 adhesive patches. In some instances, the placement area sheet is configured to hold about 8 adhesive patches. In some instances, the placement area sheet is configured to hold about 7 adhesive patches. In some instances, the placement area sheet is configured to hold about 6 adhesive patches. In some instances, the placement area sheet is configured to hold about 5 adhesive patches. In some instances, the placement area sheet is configured to hold about 4 adhesive patches. In some instances, the placement area sheet is configured to hold about 3 adhesive patches. In some instances, the placement area sheet is configured to hold about 2 adhesive patches. In some instances, the placement area sheet is configured to hold about 1 adhesive patch.

The applied patch, in some instances, is stored so that the matrix containing, skin facing surface of the applied patch is in contact with the placement area sheet. In some embodiments, a skin sample collector comprises the placement area sheet. In some embodiments, the skin sample collector is a tri-fold skin sample collector. In some instances, the tri-fold skin sample collector further comprises one or more panels. In some instances, the placement area sheet is at least one panel of the tri-fold skin sample collector. In some instances, the tri-fold skin sample collector further comprises one or more clear panels. In some instances, the tri-fold skin sample collector is labeled with a unique barcode that is assigned to a subject. In some instances, the tri-fold skin sample collector comprises an area for labeling subject information.

In an exemplary embodiment, the patch stripping method further comprises preparing the skin sample prior to application of the adhesive patch. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The patch stripping method, devices, and systems described herein are useful for the collection of a skin sample from a skin lesion. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm or from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, or from about 90 mm to about 100 mm in diameter. In some instances, the diameter is the longest diameter of the skin lesion. In some instances, the diameter is the smallest diameter of the skin lesion.

Examples of subjects include but are not limited to vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female. In some embodiments, the subject has skin previously exposed to UV light or radiation.

Such non-invasive methods in some instances provide advantages over traditional biopsy methods, including but not limited to self-application by a patient/subject, increased signal to noise ratio of sample exposed to the skin surface (leading to higher sensitivity and/or specificity), lack of temporary or permanent scarring at the analysis site, lower chance of infection, or any other advantage recognized by those of skill in the art.

A skin sample may be obtained from a subject using a collection device (such as an adhesive patch). In some embodiments of the methods described herein, a skin sample is obtained from the subject by applying an adhesive patch to a skin region of the subject. In some embodiments, the skin sample is obtained using an adhesive patch. In some embodiments, the adhesive patch comprises tape. In some embodiments, the skin sample is not obtained with an adhesive patch. In some instances, the skin sample is obtained using a brush. In some instances, the skin sample is obtained using a swab, for example a cotton swab. In some cases, the skin sample is obtained using a probe. In some cases, the skin sample is obtained using a hook. In some instances, the skin sample is obtained using a medical applicator. In some instances, the skin sample is obtained by scraping a skin surface of the subject. In some cases, the skin sample is obtained through excision. In some instances, the skin sample is biopsied. In some embodiments, the skin sample is a biopsy. In some instances, the skin sample is obtained using one or more needles. For example, the needles may be microneedles. In some instances, the biopsy is a needle biopsy, or a microneedle biopsy. In some instances, the skin sample is obtained invasively. In some instances, the skin sample is obtained noninvasively. A skin sample in some instances is obtained iteratively from the same skin area of a subject. In some instances, multiple samples are obtained from a single skin area and pooled prior to analysis.

In some instances, methods generate samples from the top or superficial layers of skin, which have been exposed to higher levels of one or more environmental factors. In some embodiments, the skin sample comprises cells of the stratum corneum. In some embodiments, the skin sample consists of cells of the stratum corneum. In some instances, non-invasive sampling described herein does not fully disrupt the epidermal and dermal junction. Without being bound by theory, non-invasive sampling described herein does not trigger significant wound healing which normally results from significant damage to the epithelial barrier. In some embodiments, the skin sample comprises at least 80%, 90%, 95%, 97%, 98%, 99%, 99.5%, or at least 99.9% of cells derived from the basal keratinocyte layer. In some embodiments, the skin sample comprises less than 10%, 5%, 3%, 2%, 1%, 0.1%, 0.05%, or less than 0.01% cells derived from the basal keratinocyte layer. In some embodiments, the skin sample does not include the basal layer of the skin. In some embodiments, the skin sample comprises or consists of a skin depth of 10 μm, 50 μm, 100 μm, 150 μm, 200 μm, 250 μm, 300 μm, 350 μm, 400 μm, 450 μm, 500 μm, or a range of skin depths defined by any two of the aforementioned skin depths. In some embodiments, the skin sample comprises or consists of a skin depth of about 10 μm, 50 μm, 100 μm, 150 μm, 200 μm, 250 μm, 300 μm, 350 μm, 400 μm, 450 μm, or about 500 μm. In some embodiments, the skin sample comprises or consists of a skin depth of 50-100 μm. In some embodiments, the skin sample comprises or consists of a skin depth of 100-200 μm. In some embodiments, the skin sample comprises or consists of a skin depth of 200-300 μm. In some embodiments, the skin sample comprises or consists of a skin depth of 300-400 μm. In some embodiments, the skin sample comprises or consists of a skin depth of 400-500 μm. In some instances the sample comprises at least 1, 5, 10, 100, 500, 1000, 5000, 10,000, 20,000, 50,000 100,000, 500,000, or at least 1 million cells. In some instances the sample comprises about 1, 5, 10, 100, 500, 1000, 5000, 10,000, 20,000, 50,000 100,000, 500,000, or about 1 million cells. In some instances the sample comprises no more than 1, 5, 10, 100, 500, 1000, 5000, 10,000, 20,000, 50,000 100,000, 500,000, or no more than 1 million cells. In some instances the sample comprises at least 1-10,000, 5-10,000, 100-10,000, 100-100,000, 100-1 million, 500-100,000, 1000-100,000, 1000-500,000, 1000-5000, 1000-10,000, 10,000-1 million, 10,000 to 500,000, 10,000 to 250,000, 10,000-100,000, 50,000-1 million, 100,000 to 1 million, or 100,000 to 5 million.

Non-invasive sampling methods described herein may comprise obtaining multiple skin samples from the same area of skin on an individual using multiple collection devices (e.g., tapes). In some instances, each sample obtained from the same area or substantially the same area results in progressively deeper layers of skin cells. In some instances, multiple samples are pooled prior to analysis.

The skin sample may be defined by thickness, or how deep into the skin cells are obtained. In some embodiments, the skin sample is no more than 10 μm thick. In some embodiments, the skin sample is no more than 50 μm thick. In some embodiments, the skin sample is no more than 100 μm thick. In some embodiments, the skin sample is no more than 150 μm thick. In some embodiments, the skin sample is no more than 200 μm thick. In some embodiments, the skin sample is no more than 250 μm thick. In some embodiments, the skin sample is no more than 300 μm thick. In some embodiments, the skin sample is no more than 350 μm thick. In some embodiments, the skin sample is no more than 400 μm thick. In some embodiments, the skin sample is no more than 450 μm thick. In some embodiments, the skin sample is no more than 500 μm thick.

In some embodiments, the skin sample is at least 10 μm thick. In some embodiments, the skin sample is at least 50 μm thick. In some embodiments, the skin sample is at least 100 μm thick. In some embodiments, the skin sample is at least 150 μm thick. In some embodiments, the skin sample is at least 200 μm thick. In some embodiments, the skin sample is at least 250 μm thick. In some embodiments, the skin sample is at least 300 μm thick. In some embodiments, the skin sample is at least 350 μm thick. In some embodiments, the skin sample is at least 400 μm thick. In some embodiments, the skin sample is at least 450 μm thick. In some embodiments, the skin sample is at least 500 μm thick.

In some embodiments, the adhesive patch removes a skin sample from the subject at a depth no greater than 10 μm. In some embodiments, the adhesive patch removes a skin sample from the subject at a depth no greater than 50 μm. In some embodiments, the adhesive patch removes a skin sample from the subject at a depth no greater than 100 μm. In some embodiments, the adhesive patch removes a skin sample from the subject at a depth no greater than 150 μm. In some embodiments, the adhesive patch removes a skin sample from the subject at a depth no greater than 200 μm. In some embodiments, the adhesive patch removes a skin sample from the subject at a depth no greater than 250 μm. In some embodiments, the adhesive patch removes a skin sample from the subject at a depth no greater than 300 μm. In some embodiments, the adhesive patch removes a skin sample from the subject at a depth no greater than 350 μm. In some embodiments, the adhesive patch removes a skin sample from the subject at a depth no greater than 400 μm. In some embodiments, the adhesive patch removes a skin sample from the subject at a depth no greater than 450 μm. In some embodiments, the adhesive patch removes a skin sample from the subject at a depth no greater than 500 μm.

In some embodiments, the adhesive patch removes 1, 2, 3, 4, or 5 layers of stratum corneum from a skin surface of the subject. In some embodiments, the adhesive patch removes a range of layers of stratum corneum from a skin surface of the subject, for example a range defined by any two of the following integers: 1, 2, 3, 4, or 5, e.g., 1-5, 2-4, 3-5, 1-3, 4-5, etc. In some embodiments, the adhesive patch removes 1-5 layers of stratum corneum from a skin surface of the subject. In some embodiments, the adhesive patch removes 2-3 layers of stratum corneum from a skin surface of the subject. In some embodiments, the adhesive patch removes 2-4 layers of stratum corneum from a skin surface of the subject. In some embodiments, the adhesive patch removes no more than the basal layer of a skin surface from the subject.

Some embodiments include collecting cells from the stratum corneum of a subject, for instance, by using an adhesive tape with an adhesive matrix to adhere the cells from the stratum corneum to the adhesive matrix. In some embodiments, the cells from the stratum corneum comprise T cells or components of T cells. In some embodiments, the cells from the stratum corneum comprise keratinocytes. In some instances, the stratum corneum comprises keratinocytes, melanocytes, fibroblasts, antigen presenting cells (Langerhans cells, dendritic cells), or inflammatory cells (T cells, B cells, eosinophils, basophils). In some embodiments, the skin sample does not comprise melanocytes. In some embodiments, a skin sample is obtained by applying a plurality of adhesive patches to a skin region of a subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a skin lesion.

Non-invasive sampling described herein may obtain amounts of nucleic acids. Such nucleic acids in some instances are obtained by collecting one or more skin samples using a single collection device. In some instances, nucleic acids are obtained from pooled samples In some instances, nucleic acids are obtained from samples pooled from multiple collection devices. In some instances, nucleic acids are obtained from samples from a single collection device applied to the skin multiple times (1, 2, 3, or 4 times). In additional embodiments, the adhered skin sample comprises cellular material including nucleic acids such as RNA, DNA, or a mix thereof, in an amount that is at least about 1 picogram. Cellular material in some instances is obtained from skin using a single collection device. In some instances, cellular material is obtained from samples pooled from multiple collection devices. In some instances, cellular material is obtained from samples from a single collection device applied to the skin multiple times (1, 2, 3, or 4 times). In some instances, cellular material is obtained from samples from a single collection device applied to the skin multiple times, in a serial fashion (i.e., serially). In some instances, an amount of cellular material described herein refers to the amount of material pooled from multiple collection devices (e.g., 1-6 devices). In some embodiments, the amount of cellular material is no more than about 1 nanogram. In further or additional embodiments, the amount of cellular material is no more than about 1 microgram. In still further or additional embodiments, the amount of cellular material is no more than about 1 milligram. In still further or additional embodiments, the amount of cellular material is no more than about 1 gram.

Following extraction of nucleic acids from a biological sample, the nucleic acids, in some instances, are further purified. In some instances, the nucleic acids are RNA. In some instances, the nucleic acids are DNA. In some instances, the nucleic acids are a combination of RNA and DNA. In some instances, the RNA is human RNA. In some instances, the DNA is human DNA. In some instances, the RNA is microbial RNA. In some instances, the DNA is microbial DNA. In some instances, cDNA is generated by reverse transcription of RNA. In some instances, human nucleic acids and microbial nucleic acids are purified from the same biological sample. In some embodiments, the biological sample, is a crude biological sample. In some embodiments, the biological sample is not further processed or manipulated prior to nucleic acid extraction. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods for isolating nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 um, 1 um, 1.5 um, 2 um, 2.5 um, 3 um, 3.5 um, 4 um, 4.5 um, 5 um, 5.5 um, 6 um, 6.5 um, 7 um, 7.5 um, 8 um, 8.5 um, 9 um, 9.5 um, 10 um, or more than 10 um.

In some cases, a yield of the nucleic acids products obtained using methods described herein is about 500 picograms or higher, about 600 picograms or higher, about 1000 picograms or higher, about 2000 picograms or higher, about 3000 picograms or higher, about 4000 picograms or higher, about 5000 picograms or higher, about 6000 picograms or higher, about 7000 picograms or higher, about 8000 picograms or higher, about 9000 picograms or higher, about 10000 picograms or higher, about 20000 picograms or higher, about 30000 picograms or higher, about 40000 picograms or higher, about 50000 picograms or higher, about 60000 picograms or higher, about 70000 picograms or higher, about 80000 picograms or higher, about 90000 picograms or higher, or about 100000 picograms or higher.

In some cases, a yield of the nucleic acids products obtained using methods described herein is about 100 picograms, 500 picograms, 600 picograms, 700 picograms, 800 picograms, 900 picograms, 1 nanogram, 5 nanograms, 10 nanograms, 15 nanograms, 20 nanograms, 21 nanograms, 22 nanograms, 23 nanograms, 24 nanograms, 25 nanograms, 26 nanograms, 27 nanograms, 28 nanograms, 29 nanograms, 30 nanograms, 35 nanograms, 40 nanograms, 50 nanograms, 60 nanograms, 70 nanograms, 80 nanograms, 90 nanograms, 100 nanograms, 150 nanograms, 200 nanograms, 250 nanograms, 300 nanograms, 400 nanograms, 500 nanograms, or higher.

In some cases, methods described herein provide less than less than 10%, less than 8%, less than 5%, less than 2%, less than 1%, or less than 0.5% product yield variations between samples.

In some embodiments, a number of cells is obtained for use in a method described herein. Some embodiments include use of an adhesive patch comprising an amount of an adhesive. Some embodiments include use of a number of adhesive patches based on the number of cells to be obtained. Some embodiments include use of an adhesive patch sized based on the number of cells to be obtained. The size and/or tackiness may be based on the type of skin to be obtained. For example, normal looking skin generally provides less cells and RNA yield than flaky skin. In some embodiments, a skin sample is used comprising skin from a subject's temple, forehead, cheek, or nose. In some embodiments, only one patch is used. In some embodiments, only one patch is applied a single time (e.g., once) to a single skin area. In some other embodiments, only one patch is applied multiple times (e.g., serially) to a single skin area. In other embodiments, a plurality of patches is applied a single time each (e.g., once) to a single skin area. In other embodiments, a plurality of patches is applied a single time each (e.g., once) to a plurality of skin areas. In yet other embodiments, a plurality of patches is applied multiple times each (e.g., serially) to a single skin area. In yet other embodiments, a plurality of patches is applied multiple times each (e.g., serially) to a plurality of skin areas. In some embodiments, only one patch is used per skin area (e.g. skin area on a subject's temple, forehead, cheek, or nose).

In some cases, methods described herein provide a substantially homogenous population of a nucleic acid product or mix of nucleic acid products. In some cases, methods described herein provide less than 30%, less than 25%, less than 20%, less than 15%, less than 10%, less than 8%, less than 5%, less than 2%, less than 1%, or less than 0.5% contaminants.

In some instances, following extraction, nucleic acids may be stored. In some instances, the nucleic acids may be stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein may be subjected to an amplification reaction following isolation and purification. In some instances, the nucleic acids to be amplified are RNA including, but not limited to, human RNA and human microbial RNA. In some instances, the nucleic acids to be amplified are DNA including, but not limited to, human DNA and human microbial DNA. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR. In some embodiments, the amplification reaction is an isothermal reaction.

Biological samples may comprise lipids. Non-limiting examples of lipids include vesicles, phospholipids, glycolipids, fatty acids, triglycerides, waxes, steroids, or other lipid. In some cases, a yield of the lipids obtained using methods described herein is 1 picogram to 100 picograms, 100 picograms to 500 picograms, 100 picograms to 1 nanogram, 1 nanogram to 1 microgram, 1 nanogram to 500 nanograms, or 500 nanograms to 5 micrograms.

Biological samples may comprise carbohydrates. Non-limiting examples of carbohydrates include sugars (e.g., monosaccharides), polysaccharides (e.g., starches), nucleotides, or fibers. In some cases, a yield of the carbohydrates obtained using methods described herein is 1 picogram to 100 picograms, 100 picograms to 500 picograms, 100 picograms to 1 nanogram, 1 nanogram to 1 microgram, 1 nanogram to 500 nanograms, or 500 nanograms to 5 micrograms.

In some instances, the layers of skin include epidermis, dermis, or hypodermis. The outer layer of epidermis is the stratum corneum layer, followed by stratum lucidum, stratum granulosum, stratum spinosum, and stratum basale. In some instances, the skin sample is obtained from the epidermis layer. In some cases, the skin sample is obtained from the stratum corneum layer. In some instances, the skin sample is obtained from the dermis. In some cases, the skin sample is obtained from the stratum germinativum layer. In some cases, the skin sample is obtained from no deeper than the stratum germinativum layer.

In some instances, cells from the stratum corneum layer are obtained, which comprises keratinocytes. In some instances, cells from the stratum corneum layer comprise T cells or components of T cells. In some cases, melanocytes are not obtained from the skin sample.

The sample may comprise skin cells from a superficial depth (superficial layer) of skin using the non-invasive sampling techniques described herein. In some instances, the superficial layer comprises about 0.01, 0.02, 0.05, 0.08, 0.1, 0.2, 0.3, 0.4 mm depth, of skin. In some instances, In some instances, the superficial layer comprises no more than about 0.01, 0.02, 0.05, 0.08, 0.1, 0.2, 0.3, 0.4 mm depth, of skin. In some instances, In some instances, the superficial layer comprises at least about 0.01, 0.02, 0.05, 0.08, 0.1, 0.2, 0.3, or at least 0.4 mm depth, of skin. In some instances, the superficial layer comprises about 0.01-0.1, 0.01-0.2, 0.02-0.1, 0.02-0.2 0.04-0.0.08, 0.02-0.08, 0.01-0.08, 0.05-0.2, or 0.05-0.1 mm depth, of skin. In some instances, the superficial layer comprises about 0.01, 0.02, 0.05, 0.08, 0.1, 0.2, 0.3, or about 0.4 m depth, of skin. In some instances, the superficial layer comprises no more than, about 0.01, 0.02, 0.05, 0.08, 0.1, 0.2, 0.3, or no more than 0.4 m depth, of skin. In some instances, the superficial layer comprises at least about 0.01, 0.02, 0.05, 0.08, 0.1, 0.2, 0.3, 0.4 m depth, of skin. I In some instances, the superficial layer comprises 0.01-0.1, 0.01-0.2, 0.02-0.1, 0.02-0.2 0.04-0.0.08, 0.02-0.08, 0.01-0.08, 0.05-0.2, or 0.05-0.1 m depth, of skin.

The sample may comprise skin cells a number of skin cell layers, for example the superficial cell layers. In some instances, the sample comprises skin cells from 1-5, 1-10, 1-20, 1-25, 1-50, 1-75, or 1-100 cell layers. In some instances, the sample comprises skin cells from about 1, 2, 3, 4, 5, 8, 10, 12, 15, 20, 22, 25, 30, 35, or about 50 cell layers. In some instances, the sample comprises skin cells from no more than 1, 2, 3, 4, 5, 8, 10, 12, 15, 20, 22, 25, 30, 35, or no more than 50 cell layers. In some instances the sample comprises at least 1, 5, 10, 100, 500, 1000, 5000, 10,000, 20,000, 50,000 100,000, 500,000, or at least 1 million cells. In some instances the sample comprises about 1, 5, 10, 100, 500, 1000, 5000, 10,000, 20,000, 50,000 100,000, 500,000, or about 1 million cells. In some instances the sample comprises no more than 1, 5, 10, 100, 500, 1000, 5000, 10,000, 20,000, 50,000 100,000, 500,000, or no more than 1 million cells. In some instances the sample comprises at least 1-10,000, 5-10,000, 100-10,000, 100-100,000, 100-1 million, 500-100,000, 1000-100,000, 1000-500,000, 1000-5000, 1000-10,000, 10,000-1 million, 10,000 to 500,000, 10,000 to 250,000, 10,000-100,000, 50,000-1 million, 100,000 to 1 million, or 100,000 to 5 million.

The sample may comprise skin cells collected from a defined skin area of the subject having a surface area. In some instances the sample comprises skin cells obtained from a skin surface area of 10-300 mm², 10-500 mm², 5-500 mm², 1-300 mm², 5-100 mm², 5-200 mm², or 10-100 mm². In some instances the sample comprises skin cells obtained from a skin surface area of at least 5, 10, 20, 25, 30, 50, 75, 90, 100, 125, 150, 175, 200, 225, 250, 275, 300, or at least 350 mm². In some instances the sample comprises skin cells obtained from a skin surface area of no more than 5, 10, 20, 25, 30, 50, 75, 90, 100, 125, 150, 175, 200, 225, 250, 275, 300, or no more than 350 mm².

Provided herein are methods of sample preparation. In some instances such methods transform a biological sample from a patient into a biological sample useful for predicting a response to a disease or condition having cutaneous manifestations. In some instances, a method for preparing a nucleic acid sample from a subject useful for predicting a response to a disease or condition having cutaneous manifestations comprising one or more steps of: extracting nucleic acids and/or proteins from a first biological sample of a subject, wherein the nucleic acids are obtained from the first biological sample using a non-invasive or minimally invasive sampling technique; excising a second biological sample from the subject; applying one or more treatments to the second biological sample for a time period, wherein the treatments are applied in-vitro; extracting nucleic acids and/or proteins from the second biological sample; measuring a signature for the first biological sample to generate a baseline signature; measuring a signature for the second biological sample to generate a treatment signature; comparing the baseline signature and the treatment signature to generate an outcome signature corresponding to the one or more treatments. In some instances, the sample preparation method comprises 1, 2, 3, 4, 5, 6, or more than 6 steps. In some instances, the skin biopsy sample is contacted with keratinocyte basal medium. In some instances, the method comprises detection of nucleic acids corresponding to genes measured in the treatment signature. In some instances, the method comprises detection of proteins measured in the treatment signature. In some instances, the method comprises detection of lipids measured in the treatment signature. In some instances, the method comprises detection of metabolites measured in the treatment signature. In some instances, the first biological sample comprises cellular material from the stratum corneum which has been separated from the remainder of epidermis. In some instances, the second biological sample comprises cellular material from the epidermis. In some instances, the second biological sample is obtained from a skin biopsy. In some instances, comparing comprises correlating the presence or absence of one or more biomarkers from the first biological sample and the second biological sample. In some instances, comparing comprises correlating the abundance of one or more biomarkers from the first biological sample and the second biological sample. In some embodiments, the first and/or second biological sample is a crude biological sample.

### Biomarker Signatures

Disclosed herein are methods for identifying and measuring biomarkers associated with diseases or conditions having cutaneous manifestations described herein. In some instances, measuring biomarkers results is used to generate biomarker signatures. In some embodiments, the method comprises identifying and measuring at least one biomarker for predicting therapeutic response or outcome. In some embodiments, a baseline biomarker signature may be determined at least based on the identifying and measuring the at least one biomarker. In some embodiments, a treatment biomarker signature may be determined at least based on the identifying and measuring the at least one biomarker. In some embodiments, an outcome signature may be determined at least based on the identifying and measuring the at least one biomarker. In some embodiments, the biomarker signature comprises a nucleic acid (e.g., genotypic biomarker, a single nucleotide polymorphism biomarker, a gene mutation biomarker, a gene copy number biomarker, a DNA methylation biomarker, a DNA acetylation biomarker, a chromosome dosage biomarker, a gene expression biomarker), a protein (e.g., protein expression, protein activation), a lipid, a carbohydrate, a metabolite, or a combination thereof. In some embodiments, biomarkers comprise nucleic acid mutations present in genetic material of a sample obtained from a subject. In some instances, methods described herein quantify the mutations of a sample obtained from a subject. In some embodiments, such biomarkers comprise nucleic acid expression levels. The nucleic acid may be gene-coding nucleic acid such as mRNA. In some embodiments, the nucleic acid is non-coding nucleic acid such as miRNA. The methods and devices provided herein, in certain embodiments, involve measuring or identifying biomarkers obtained from biological samples. In some instances, biological samples comprise one or more of nucleic acids, lipids, carbohydrates, or proteins. In some instances, one or more biomarkers are used to generate a biomarker signature. In some instances, the biomarker signature is a baseline signature obtained prior to treatment of the biological sample. In some instances, the biomarker signature is a treatment signature obtained subsequent to treatment of the biological sample. In some instances, the nucleic acid comprises RNA, DNA, or a combination thereof.

Described herein, in some embodiments, are methods for assaying biological samples. In some embodiments, the assaying of the biological samples may at least partially determine the signatures described herein. In some cases, the biological samples may be obtained directly from the subject. For example, the biological sample may comprise liquid biopsy such as serum and plasma or skin biopsy obtained from the subject. In some cases, the biological sample may comprise biomolecules such as nucleic acid, protein (e.g., cytokines secreted by the cultured skin biopsy sample described herein), or lipid such as ceramides (CERs), cholesterol, or free fatty acids (FFAs). Biological samples may be stored in a variety of storage conditions before processing, such as at different temperatures (e.g., at room temperature, under refrigeration or freezer conditions, at 25° C., at 4° C., at −18° C., −20° C., or at −80° C.) or as various suspensions in a collection receptacle (e.g., EDTA collection tubes, RNA collection tubes, or DNA collection tubes).

After obtaining a biological sample from a subject, the biological sample may be processed to generate biomarker signatures. For example, a presence, absence, or quantitative assessment of nucleic acid molecules within the biological sample for a panel of disease or condition associated genomic loci (e.g., quantitative measures of RNA transcripts such as mRNA and microRNA or DNA at the disease or condition associated genomic loci), proteomic data comprising quantitative measures of proteins of the dataset for a panel of disease or condition associated proteins, and/or metabolome data comprising quantitative measures for a panel of disease or condition associated metabolites may be indicative of the presence or severity of the disease or condition. Processing the biological sample obtained from the subject may comprise (i) subjecting the biological sample to conditions that are sufficient to isolate, enrich, or extract a plurality of nucleic acid molecules, proteins, and/or metabolites, and (ii) assaying the plurality of nucleic acid molecules, proteins, and/or metabolites to generate the dataset. In some instances, the disease comprises cutaneous manifestations. In some instances, the disease comprises a dermatological disease.

In some embodiments, a plurality of nucleic acid molecules is extracted from the biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the biological sample by a variety of methods, such as a FastDNA Kit protocol from MP Biomedicals, a QIAamp DNA biological mini kit from Qiagen, or a biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with disease related states. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a biological sample may be tagged, e.g., with identifiable tags, to allow for multiplexing of a plurality of samples. Any number of RNA or DNA samples may be multiplexed. For example a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial biological samples. For example, a plurality of biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the disease related state. For example, the sequence reads may be aligned to one or more reference genomes (e.g., a genome of one or more species such as a human genome). The aligned sequence reads may be quantified at one or more genomic loci to generate the datasets indicative of the disease related state. For example, quantification of sequences corresponding to a plurality of genomic loci associated with disease related states may generate the datasets indicative of the disease related state.

The biological sample may be processed without any nucleic acid extraction. For example, the disease related state may be identified or monitored in the subject by using probes configured to selectively enrich nucleic acid (e.g., RNA or DNA) molecules corresponding to the plurality of disease or condition associated genomic loci. The probes may be nucleic acid primers. The probes may have sequence complementarity with nucleic acid sequences from one or more of the plurality of disease or condition associated genomic loci or genomic regions. The plurality of disease or condition associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct disease or condition associated genomic loci or genomic regions. The plurality of disease or condition associated genomic loci or genomic regions may comprise one or more members (e.g., 1, 2, 3, 4, 5, 6, 7, 8, 9, 10, 11, 12, 13, 14, 15, 16, 17, 18, 19, 20, about 25, about 30, about 35, about 40, about 45, about 50, about 55, about 60, about 65, about 70, about 75, about 80, or more) selected from the any one of the genes described herein.

The probes may be nucleic acid molecules (e.g., RNA or DNA) having sequence complementarity with nucleic acid sequences (e.g., RNA or DNA) of the one or more genomic loci (e.g., disease or condition associated genomic loci). These nucleic acid molecules may be primers or enrichment sequences. The assaying of the biological sample using probes that are selective for the one or more genomic loci (e.g., disease or condition associated genomic loci) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more genomic loci (e.g., disease or condition associated genomic loci) to generate the data indicative of the disease related state. For example, quantification of array hybridization or polymerase chain reaction (PCR) corresponding to a plurality of genomic loci (e.g., disease or condition associated genomic loci) may generate data indicative of the disease related state. Assay readouts may comprise quantitative PCR (qPCR) values, digital PCR (dPCR) values, digital droplet PCR (ddPCR) values, fluorescence values, etc., or normalized values thereof.

A biomarker signature may be quantitative (e.g., numeric or alphanumeric), with higher or lower resolution (e.g., 1-10 or high/medium/low), or qualitative (e.g., significant increase/decrease relative to a cohort), or the like. In some embodiments, the biomarker signature is quantitative. In some embodiments, the biomarker signature is numeric. In some embodiments, the biomarker signature is alphanumeric. In some embodiments, the biomarker signature is alphabetic. In some embodiments, the biomarker signature is a value or a range of values such as 1-10 or A-Z. In some embodiments, the biomarker signature is relative or general, for example: “low,” “medium,” or “high.” In some embodiments, the biomarker signature is relative to a control biomarker signature, or relative to a baseline (e.g. pre-exposure) biomarker signature.

In some embodiments, biomarker signatures are weighted (e.g., based on type of biomarker, frequency, amount of expression/concentration, ability to predict a treatment outcome, or other factor). In some embodiments, the weight of the biomarker signatures is compared to a threshold. In some embodiments, the weight of a biomarker signatures is assigned by a computer algorithm. In some embodiments, the biomarker signatures of a biomarker affects how much a particular biomarker contributes to calculating am biomarker signature, such as an outcome signature. In some embodiments, the weight of a first biomarker is less than the weight of a second biomarker. In such cases, the first biomarker may be less informative of the outcome signature than the second mutation. In some embodiments, the weight of a first biomarker is greater than the weight of a second biomarker level. In some embodiments, each biomarker is given a separate weight in the mathematical algorithm. For example, one biomarker may have a greater impact on the biomarker signature than another mutation.

In some embodiments, the weight is 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 50, or 100, in relation to another of the mutations. In some embodiments, the weight is 0.01-0.1 in relation to another of the mutations. In some embodiments, the weight is 0.1-0.5 in relation to another of the mutations. In some embodiments, the weight is 0.5-1 in relation to another of the mutations. In some embodiments, the weight is 1-1.5 in relation to another of the mutations. In some embodiments, the weight is 1.5-2 in relation to another of the mutations. In some embodiments, the weight is 2-10 in relation to another of the mutations. In some embodiments, the weight is 10-100 in relation to another of the mutations. In some embodiments, the mutations is weighted such that it contributes 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 50, or 100% of the biomarker signature.

### Genetic Mutations

Disclosed herein are methods for determining signatures based on quantifying mutations from biological samples. In some instances, a baseline, treatment, or outcome signature comprises one or more mutations. In some embodiments, a baseline biomarker signature comprises one or more mutations. In some embodiments, a treatment biomarker signature may be determined based on one or more mutations. In some cases, the skins samples may be obtained by the kits and methods described herein. In some embodiments, the skin samples may be obtained by the non-invasive (e.g. the adhesive tape) methods described herein. In some instances, a identifying biomarkers comprises determining the presence of one or more mutations. In some instances, mutations are present in genomic DNA. In some instances, mutations comprise substitutions, deletions, or additions. In some instances, mutations are present in coding regions. In some instances, mutations are present in non-coding regions. In some instances, mutations are present in genes. In some instances, mutations are present in transcription factors binding sites, promoters, terminators or other regulatory element. In some instances mutations are present in the same gene. In some instances, mutations are present in multiple genes. In some instances, genetic mutations are obtained using non-invasive sampling techniques. In some cases, the genetic mutations may be multiple mutations in a single skin sample. For example, multiple mutations may be measured, detected, or used in the methods described herein. Some embodiments include quantifying biomarkers based on multiple mutations. Some embodiments include quantifying biomarkers based on a first mutation and based on a second mutation.

Mutations may be present at any abundance in a given cell population. In some instances, the cell population is comprised of different cell types. In some instances, mutations are analyzed as a function of specific cell types. In some instances, the cell population is comprised of keratinocytes, melanocytes, fibroblasts, antigen presenting cells (e.g., Langerhans cells, or dendritic cells), and/or inflammatory cells (e.g., T cells or B cells). In some instances, the cell population is comprised of at least one of keratinocytes, melanocytes, fibroblasts, antigen presenting cells (e.g., Langerhans cells or dendritic cells), or inflammatory cells (e.g., T cells or B cells). In some instances, the cell population comprises a comparator sample. In some instances, a comparator sample is a bulk sample from a population of individuals, a sample which has been exposed to none or low amounts of an environmental factor in the same or different individual, or a sample obtained from a different area of skin on the same or different individual. The abundance of a mutation in a sample in some instances is expressed as a percentage of cells comprising the mutation or a ratio of cells comprising the mutation to cells without the mutation from the same cell type, skin location, individual, or sample. In some instances, a mutation is present at a rate in the cells of the sample. In some instances, a mutation is present at a rate of about 10%, 8%, 6%, 5%, 4% 3%, 2%, 1%, 0.5%, 0.2%, 0.1%, 0.08%, 0.05%, or about 0.01%. In some instances, a mutation is present at a rate of at least 10%, 8%, 6%, 5%, 4% 3%, 2%, 1%, 0.5%, 0.2%, 0.1%, 0.08%, 0.05%, or at least 0.01%. In some instances, a mutation is present at a rate of no more than 10%, 8%, 6%, 5%, 4% 3%, 2%, 1%, 0.5%, 0.2%, 0.1%, 0.08%, 0.05%, or no more than 0.01%. In some instances, a mutation is present at a rate of 1%-5%, 1%-4%, 1%-3%, 0.5%-5%, 0.5%-1%, 0.5%-2%, 2%-10%, 5%-10%, or 4%-10%. In some instances, a mutation is present in a sample at a ratio of the number of cells comprising a mutation relative to the number of total cells in the sample (e.g., mutations/cell). In some instances, a mutation is present in a sample at a ratio of at least 1:5, 1:10, 1:15, 1:20, 1:50, 1:70, 1:100, or 1:200. In some instances, a mutation is present in a sample at a ratio of no more than 1:5, 1:5, 1:15, 1:20, 1:50, 1:70, 1:100 or 1:200. In some instances, a mutation is present in a sample at a ratio of 1:3-1:100, 1:5-1:100, 1:10-1:100, 1:20-1:500, 1:20-1:200, 1:20-1:100, 1:20-1:200, or 1:30-1:200. In some instances, the abundance of a mutation determines the sensitivity needed to detect the mutation. In some instances, the methods described herein detect mutations with a sensitivity of about 0.1%, 0.2%, 0.5%, 1%, 1.5%, 2%, 3%, 4%, 5%, 7%, 10%, or about 15%. In some instances, the methods described herein detect mutations with a sensitivity of at least 0.1%, 0.2%, 0.5%, 1%, 1.5%, 2%, 3%, 4%, 5%, 7%, 10%, at least 15%. In some instances, the methods described herein detect mutations with a sensitivity of no more than 0.1%, 0.2%, 0.5%, 1%, 1.5%, 2%, 3%, 4%, 5%, 7%, 10%, or no more than 15%. In some instances, the methods described herein detect mutations with a sensitivity of about 0.1%-10%, 0.1-1%, 0.5-5%, 0.5-3%, 1%-10%, 1%-5%, 0.5-20%, or 1%-15%.

Mutations may be present in a gene at any copy number in a cell. In some instances, a mutation is present in a gene at one, two, three, four, five, six, seven, ten, or even more than 10 copies in a cell. In some instances, a mutation is present in a gene in at least two copies in a cell. Mutations may be present in a gene at any allele frequency in a cell. In some instances, a mutation is present at an allele frequency of at one, two, three, four, five, six, seven, ten, or even more than 10 copies in a cell. In some instances, a mutation is present at an allele frequency of at least two copies in a cell.

In some embodiments, the genetic mutations may include more than one mutation. For example, the method may include measuring, detecting, receiving, or using mutations. In some embodiments, detecting comprises determining the presence or absence of one or more mutations. Some embodiments include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more mutations. Some embodiments include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more mutations, or a range of mutations defined by any two of the aforementioned integers. For example, some embodiments include measuring the frequency of about 10 mutations. Some embodiments include measuring the frequency of about 20 mutations. Some embodiments include measuring the frequency of about 30 mutations. Some embodiments include measuring the frequency of about 40 mutations. Some embodiments include measuring the frequency of 50 mutations. Some embodiments include measuring the frequency of 1-4 mutations. Some embodiments include measuring the frequency of 1-7 mutations. Some embodiments include measuring the frequency of 1-10 mutations. Some embodiments include measuring the frequency of 1-100 mutations. Some embodiments include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 mutations. Some embodiments include no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 mutations.

Mutations described herein may be measured using any method known in the art. In some instances, mutations are identified using PCR. In some instances, mutations are identified using Sanger sequencing. In some instances, mutations are identified using Next Generation Sequencing or sequencing by synthesis. In some instances, mutations are identified using nanopore sequencing. In some instances, mutations are identified using real time PCR (qPCR). In some instances, mutations are identified using digital PCR (ddPCR). In some instances, mutations are identified using mass analysis. In some instances, 10, 100, 1000, 10,000, or more than 10,000 samples are assayed in parallel.

Mutations described herein may be present in a gene. In some instances, the gene is a gene which drives increased cell proliferation. In some instances, the gene is TP53, NOTCH1, NOTCH2, NOTCH3, RBM10, PPP2R1A, GNAS, CTNNB1, PIK3CA, PPP6C, HRAS, KRAS, MTOR, SMAD3, LMNA, FGFR3, ZNF750, EPAS1, RPL22, ALDH2, CBFA2T3, CCND1, FAT1, FH, KLF4, CIC, RAC1, PTCH1, or TPM4. In some instances, the mutation is a C to T or G to A substitution.

In some embodiments, the one or more mutations are present in a MAPK pathway gene. In some embodiments, the MAPK pathway gene includes but is not limited to BRAF, CBL, MAP2K1, NF1, or RAS.

The at least one mutation may be present in an MTOR pathway gene. In some embodiments, the MTOR pathway gene includes but is not limited to MTOR, AKT, AKT1 (v-akt murine thymoma viral oncogene homolog 1), AKT1S1 (AKT1 substrate 1 (proline-rich)), ATG13 (autophagy related 13), BNIP3 (BCL2/adenovirus E1B 19 kDa interacting protein 3), BRAF (B-Raf proto-oncogene, serine/threonine kinase), CCNE1 (cyclin E1), CDK2 (cyclin-dependent kinase 2), CLIP1 (CAP-GLY domain containing linker protein 1), CYCS (cytochrome c, somatic), DDIT4 (DNA-damage-inducible transcript 4), DEPTOR (DEP domain containing MTOR-interacting protein), EEF2 (eukaryotic translation elongation factor 2), EIF4A1 (eukaryotic translation initiation factor 4A1), EIF4B (eukaryotic translation initiation factor 4B), EIF4E (eukaryotic translation initiation factor 4E), EIF4EBP1 (eukaryotic translation initiation factor 4E binding protein 1), FBXW 11 (F-box and WD repeat domain containing 11), HRAS (Harvey rat sarcoma viral oncogene homolog), IKBKB (inhibitor of kappa light polypeptide gene enhancer in B-cells, kinase beta), IRS1 (insulin receptor substrate 1), MAP2K1 (mitogen-activated protein kinase 1), MAP2K2 (mitogen-activated protein kinase 2), MAPK1 (mitogen-activated protein kinase 1), MAPK3 (mitogen-activated protein kinase 3), MAPKAP1 (mitogen-activated protein kinase associated protein 1), MLST8 (MTOR associated protein, LST8 homolog), MTOR (mechanistic target of rapamycin (serine/threonine kinase)), NRAS (neuroblastoma RAS viral (v-ras) oncogene homolog), PDCD4 (programmed cell death 4 (neoplastic transformation inhibitor)), PDPK1 (3-phosphoinositide dependent protein kinase 1), PLD1 (phospholipase D1, phosphatidylcholine-specific), PLD2 (phospholipase D2), PML (promyelocytic leukemia), POLDIP3 (polymerase (DNA-directed), delta interacting protein 3), PPARGC1A (peroxisome proliferator-activated receptor gamma, coactivator 1 alpha), PRKCA (protein kinase C, alpha), PRR5 (proline rich 5 (renal)), PXN (paxillin), RAC1 (ras-related C3 botulinum toxin substrate 1 (rho family, small GTP binding protein Rac1)), RAF1 (Raf-1 proto-oncogene, serine/threonine kinase), RBICC1 (RB1-inducible coiled-coil 1), RHEB (Ras homolog enriched in brain), RHOA (ras homolog family member A), RICTOR (RPTOR independent companion of MTOR, complex 2), RPS6KA1 (ribosomal protein S6 kinase, 90 kDa, polypeptide 1), RPS6KB1 (ribosomal protein S6 kinase, 70 kDa, polypeptide 1), RPTOR (regulatory associated protein of MTOR, complex 1), RRAGA (Ras-related GTP binding A), RRAGB (Ras-related GTP binding B), RRAGC (Ras-related GTP binding C), RRAGD (Ras-related GTP binding D), RRN3 (RRN3 RNA polymerase I transcription factor homolog), SFN (stratifin), SGK1 (serum/glucocorticoid regulated kinase 1), SREBF1 (sterol regulatory element binding transcription factor 1), SSPO (SCO-spondin), TSC1 (tuberous sclerosis 1), TSC2 (tuberous sclerosis 2), ULK1 (unc-51 like autophagy activating kinase 1), ULK2 (unc-51 like autophagy activating kinase 2), YWHAB (tyrosine 3-monooxygenase/tryptophan 5-monooxygenase activation protein, beta), YWHAE (tyrosine 3-monooxygenase/tryptophan 5-monooxygenase activation protein, epsilon), YWHAG (tyrosine 3-monooxygenase/tryptophan 5-monooxygenase activation protein, gamma), YWHAH (tyrosine 3-monooxygenase/tryptophan 5-monooxygenase activation protein, eta), YWHAQ (tyrosine 3-monooxygenase/tryptophan 5-monooxygenase activation protein, theta), YWHAZ (tyrosine 3-monooxygenase/tryptophan 5-monooxygenase activation protein, zeta), or YY1 (YY1 transcription factor).

In some embodiments, the at least one mutation is present in MTOR. In some embodiments, the at least one mutation in MTOR comprises S2215F. In some embodiments, the at least one mutation in MTOR comprises c.6644C>T.

The at least one mutation may be present in an HRAS pathway gene. In some embodiments, the HRAS pathway gene includes but is not limited to HRAS. In some embodiments, the at least one mutation is present in HRAS. In some embodiments, the at least one mutation in HRAS comprises G12D, Q61L, or G13D. In some embodiments, the at least one mutation in HRAS comprises c.35G>A, c.182A>T, or c.38G>A.

In some embodiments, the one or more mutations are present in an RNA processing gene. In some embodiments, the RNA processing gene includes but is not limited to DDX3X.

In some embodiments, the one or more mutations are present in a PI3K pathway gene. In some embodiments, the one or more mutations are present in a PI3KCA family gene. In some instances, the PI3KCA family gene includes but is not limited to XIAP (BIRC4) (X-linked inhibitor of apoptosis), AKT1 (v-akt murine thymoma viral oncogene homolog 1), TWIST1 (Twist homolog 1 (Drosophila)), BAD (BCL2-associated agonist of cell death), CDKN1A (p21) (Cyclin-dependent kinase inhibitor 1A (p21, Cip1))), ABL1 (v-abl Abelson murine leukemia viral oncogene homolog 1), CDH1 (Cadherin 1, type 1, E-cadherin), TP53 (Tumor protein p53), CASP3 (Caspase 3, apoptosis-related cysteine peptidase), PAK1 (p²1/Cdc42/Rac1-activated kinase 1), GAPDH (Glyceraldehyde-3-phosphate dehydrogenase), PIK3CA (Phosphoinositide-3-kinase, catalytic, α-polypeptide), FAS (TNF receptor superfamily, member 6), AKT2 (v-akt murine thymoma viral oncogene homolog 2), FRAP1 (mTOR) (FK506 binding protein 12-rapamycin associated protein 1), FOXO1A (Forkhead box 01), PTK2 (FAK) (PTK2 protein tyrosine kinase 2), CASP9 (Caspase 9, apoptosis-related cysteine peptidase), PTEN (Phosphatase and tensin homolog), CCND1 (Cyclin D1), NFKB1 (Nuclear factor x-light polypeptide gene enhancer B-cells 1), GSK3B (Glycogen synthase kinase 3-0), MDM2 (Mdm2 p53 binding protein homolog (mouse)), or CDKN1B (p27) (Cyclin-dependent kinase inhibitor 1B (p27, Kip1)).

In some embodiments, the one or more mutations are present in a chromatin remodeling gene. In some embodiments, the chromatin remodeling gene includes but is not limited to ARID2.

In some embodiments, the one or more mutations are present in a transcription regulation region of a gene. In some embodiments, the region comprises a promoter. In some embodiments, the region comprises a terminator. In some embodiments, the region comprises a Kozak consensus sequence, stem loop structures or internal ribosome entry site. In some instances, the region comprises an enhancer, a silencer, an insulator, an operator, aa promoter, a 5′ untranslated region (5′ UTR), or a 3′ untranslated region (3′UTR).

Mutations described herein may be identified phenotypically. In some instances, mutations are identified using staining techniques. In some instances, the staining technique is an immunogenic staining technique. In some instances, samples comprise cells having p53 immunopositive patches (PIPs). In some instances, the one or more mutations are present in PIPs.

In some cases, the mutations described herein may include a cytokine or inflammatory protein or a receptor of the cytokine of the inflammatory protein. Exemplary cytokine or inflammatory protein may include 4-1BBL, acylation stimulating protein, adipokine, albinterferon, APRIL, Arh, BAFF, Bcl-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MIDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-17A, IL-18, IL-18BP, IL-19, IL-1A, IL-1B, IL4, IL4R alpha, IL-1F10, IL-13, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL, 23p19, IL-24, IL-28A, IL-28B, IL-29, IL-3, IL-31, IL-33, IL-35, IL-4, IL-5, IL-6, IL-6-like, IL-7, IL-8/CXCL8, IL-9, inflammasome, interferome, interferon, interferon beta-1a, interferon beta-1b, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFα (TNF alpha), TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2.

### Epigenetics

Epigenetic markers may be evaluated alone, or in combination with mutations for determining the signatures described herein. In some instances, a quantified burden is generated from at least one epigenetic marker. In some instances, the epigenetic markers an genomic modification. In some instances, the at least one genomic modification comprises methylation in a CpG island of a gene or a transcription regulation region of the gene. In some instances, the at least one epigenetic marker comprises 5-methylcytosine (“methylation”). In some instances, the at least one genomic modification comprises N6-methyladenine. In some instances, an epigenetic marker comprises chromatin remodeling. In some instances, chromatic remodeling comprises modification of histones. In some instances, modification of histones comprises methylation, acetylation, phosphorylation, ubiquitination, sumoylation, citrullination, or ADP-ribosylation. In some instances, the at least one genomic modification is correlated with increased exposure to environmental factors. In some instances, the at least one genomic modification is correlated with at least one additional genetic mutation.

Epigenetic markers may be found within specific genes, near genes (e.g., promoter, terminator), or outside of genes. In some instance, at least one epigenetic markers is present in a keratin family gene. In some instances, the epigenetic marker is a proliferative marker in inflammatory diseases. In some instance, at least one epigenetic marker is present in KRT1, KRT5, KRT6, KRT14, KRT15, KRT16, KRT17, or KRT80.

Numerous methods are known in the art for resolving epigenetic markers. In some embodiments, the epigenetic markers is methylation of cytosine. In some instances, methylation sensitive endonucleases are used to identify such modifications. In some instances chemical or enzymatic differentiation of methylated vs. unmethylated bases is used (e.g., methyl C conversion to U using bisulfite). After conversion and comparison to untreated samples, methylation patterns are in some instances obtained using various sequencing and analysis techniques described herein.

Mutations in samples may be processed or analyzed in parallel using high-throughput multiplex methods described herein to identify biomarker signatures (e.g., mass-array, hybridization array, specific probe hybridization, whole genome sequencing, or other method). In some embodiments, methods described herein comprise genotyping. The nucleic acids analyzed from the sample in some instances represent the entire genome or a sub-population thereof (e.g., genomic regions, genes, introns, exons, promoters, intergenic regions). In some instances, these nucleic acids are analyzed from one or more panels which target mutations or groups of mutations. In some instances, methods describe herein comprise detecting one or more mutations in these nucleic acids. In some instances, 25-50,000, 50-50,000, 100-100,000, 25-10,000, 25-5,000 or 300-700 mutations are analyzed. In some instances, at least 300, 400, 500, 750, 1000, 2000, 5000, 10,000, or more than 10,000 mutations are analyzed. In some instances, two or more mutations are used to generate a pattern or profile representative of the biomarker signature. In some examples, a subset of genomic regions will be sequenced to perform a panel analysis of mutations in the subset of genomic regions (or of the whole genome) to output a set of mutations for the sample. For instance, a variety of mutational panels could be utilized, for instance the MSK-TMPACT panel. Accordingly, the result of this process in some instances is an output of a set of mutations based on the subset of sequenced genomic regions or the whole genome. In some instances, the sequence data is transmitted over a network to be stored in a database by a server or further processed on local memory. In some examples, the server may then perform further processing on the sequence data or sequence data files.

RNA Expression Levels

Biomarkers may comprise genes (or gene classifiers) and expression levels thereof. In some instances, a baseline, treatment, or outcome signature comprises a gene signature. In some instances, expression levels of genes are obtained through analysis of nucleic acids, such as RNA. In some instances, the expression level of a gene associated with a disease or condition having cutaneous manifestations is a biomarker.

A biomarker may comprise a gene associated with skin cancer. In some instances, methods herein comprise measuring the expression level of a gene associated with skin cancer. In one embodiment, the gene is any one or more of interferon regulatory factor 6, claudin 23, melan-A, osteopetrosis associated transmembrane protein 1, RAS-like family 11 member B, actinin alpha 4, transmembrane protein 68, Glycine-rich protein (GRP3 S), Transcription factor 4, hypothetical protein FLJ20489, cytochrome c somatic, transcription factor 4, Forkhead box P1, transducer of ERBB2-2, glutaminyl-peptide cyclotransferase (glutaminyl cyclase), hypothetical protein FLJ10770, selenophosphate synthetase 2, embryonal Fyn-associated substrate, Kruppel-like factor 8, Discs large homolog 5 (Drosophila), regulator of G-protein signalling 10, ADP-ribosylation factor related protein 2, TIMP metallopeptidase inhibitor 2, 5-aminoimidazole-4-carboxamide ribonucleotide formyltransferase/IMP cyclohydrolase, similar to RIKEN cDNA 5730421E18 gene, Regulator of G-protein signalling 10, Nuclear RNA-binding protein putative, tyrosinase-related protein 1, TIMP metallopeptidase inhibitor 2, Claudin 1, transcription factor 4, solute carrier family 16 (monocarboxylic acid transporters) member 6 (similar to solute carrier family 16 member 6; monocarboxylate transporter 6), PTCH1, PTCH2, CDKN2A, CDK4, MITF, BAP1, BRCA2, or any combination thereof. In some cases, the expression levels are measured by contacting the isolated nucleic acids with an additional set of probes that recognizes interferon regulatory factor 6, claudin 23, melan-A, osteopetrosis associated transmembrane protein 1, RAS-like family 11 member B, actinin alpha 4, transmembrane protein 68, Glycine-rich protein (GRP3 S), Transcription factor 4, hypothetical protein FLJ20489, cytochrome c somatic, transcription factor 4, Forkhead box P1, transducer of ERBB2-2, glutaminyl-peptide cyclotransferase (glutaminyl cyclase), hypothetical protein FLJ10770, selenophosphate synthetase 2, embryonal Fyn-associated substrate, Kruppel-like factor 8, Discs large homolog 5 (Drosophila), regulator of G-protein signalling 10, ADP-ribosylation factor related protein 2, TIMP metallopeptidase inhibitor 2, 5-aminoimidazole-4-carboxamide ribonucleotide formyltransferase/IMP cyclohydrolase, similar to RIKEN cDNA 5730421E18 gene, Regulator of G-protein signalling 10, Nuclear RNA-binding protein putative, tyrosinase-related protein 1, TIMP metallopeptidase inhibitor 2, Claudin 1, transcription factor 4, solute carrier family 16 (monocarboxylic acid transporters) member 6 (similar to solute carrier family 16 member 6; monocarboxylate transporter 6), PTCH1, PTCH2, CDKN2A, CDK4, MITF, BAP1, BRCA2, or any combination thereof.

A biomarker may comprise a gene associated with atopic dermatitis. In some instances, methods herein comprise measuring the expression level of a gene associated with atopic dermatitis. In some instances, the gene comprises Interleukin 13 (IL-13), Interleukin 31 (IL-31), Thymic Stromal Lymphopoietin (TSLP), IL4Ralpha, or a combination thereof. In some embodiments, the gene comprises Interleukin 13 Receptor (IL-13R), Interleukin 4 Receptor (IL-4R), Interleukin 17 (IL-17), Interleukin 22 (IL-22), C-X-C Motif Chemokine Ligand 9 (CXCL9), C-X-C Motif Chemokine Ligand 10 (CXCL10), C-X-C Motif Chemokine Ligand 10 (CXCL11), S100 Calcium Binding Protein A7 (S100A7), S100 Calcium Binding Protein A8 (S100A8), S100 Calcium Binding Protein A9 (S100A9), C-C Motif Chemokine Ligand 17 (CCL17), C-C Motif Chemokine Ligand 18 (CCL18), C-C Motif Chemokine Ligand 19 (CCL19), C-C Motif Chemokine Ligand 26 (CCL26), C-C Motif Chemokine Ligand 27 (CCL27), Nitric Oxide Synthetase 2 (NOS2) or a combination thereof. In some cases, the expression levels are measured by contacting the isolated nucleic acids with an additional set of probes that recognizes IL-13R, IL-4R, IL-17, IL-22, CXCL9, CXCL10, CXCL11, S100A7, S100A8, S100A9, CCL17, CCL18, CCL19, CCL26, CCL27, NOS2, or a combination thereof, and detects binding between IL-13R, IL-4R, IL-17, IL-22, CXCL9, CXCL10, CXCL11, S100A7, S100A8, S100A9, CCL17, CCL18, CCL19, CCL26, CCL27, NOS2, or a combination thereof and the additional set of probes.

A biomarker may comprise a gene or gene classifier associated with psoriasis. In some instances, methods herein comprise measuring the expression level of a gene associated with psoriasis. In some instances, the gene comprises Interleukin 17A (IL-17A), Interleukin 17F (IL-17F), Interleukin 8 (IL-8), C-X-C Motif Chemokine Ligand 5 (CXCL5), S100 Calcium Binding Protein A9 (S100A9), Defensin Beta 4A (DEFB4A), TNFalpha, or a combination thereof. In some embodiments, the method further comprises detecting the expression levels of Interleukin 17C (IL-17C), S100 Calcium Binding Protein A7 (S100A7), Interleukin 17 Receptor A (IL-17RA), Interleukin 17 Receptor C (IL-17RC), Interleukin 23 Subunit Alpha (IL-23A), Interleukin 22 (IL-22), Interleukin 26 (IL-26), Interleukin 24 (IL-24), Interleukin 6 (IL-6), C-X-C Motif Chemokine Ligand 1 (CXCL1), Interferon Gamma (IFN-gamma), Interleukin 31, (IL-31), Interleukin 33 (IL-33), Tumor Necrosis Factor (TNFα), Lipocalin 2 (LCN2), C-C Motif Chemokine Ligand 20 (CCL20), TNF Receptor Superfamily Member 1A (TNFRSF1A) or a combination thereof. In some cases, measuring gene expression levels comprises contacting the isolated nucleic acids with an additional set of probes that recognizes IL-17C, S100A7, IL-17RA, IL-17RC, IL-23A, IL-22, IL-26, IL-24, IL-6, CXCL1, IFN-gamma, IL-31, IL-33, TNFα, LCN2, CCL20, TNFRSF1A, or a combination thereof, and detects binding between IL-17C, S100A7, IL-17RA, IL-17RC, IL-23A, IL-22, IL-26, IL-24, IL-6, CXCL1, IFN-gamma, IL-31, IL-33, TNFα, LCN2, CCL20, TNFRSF1A, or a combination thereof and the additional set of probes.

A biomarker may comprise a gene associated with lupus. In some instances, methods herein comprise measuring the expression level of a gene associated with lupus erythematosus. In some instances, the gene comprises Interferon Alpha 1 (IFNA1), Interferon Alpha 2 (IFNA2), Interferon Alpha 4 (IFNA4), Interferon Alpha And Beta Receptor Subunit 1 (IFNR1), Interferon Alpha And Beta Receptor Subunit 2 (IFNR2), C-C Motif Chemokine Ligand 5 (CCL5), or a combination thereof. In some embodiments, measuring expression levels of Interferon Beta 1 (IFNB1), Interferon Epsilon (IFNE), Interferon Omega 1 (IFNW1), Adenosine Deaminase, RNA Specific (ADAR), Interferon Induced proteins with Tetratricopeptide repeat (IFIT), interferon-inducible p200 family of proteins (IFI), Interferon Regulatory Factors (IRF), 2′-5′-Oligoadenylate Synthetase 1 (OAS1), Interleukin 1 Receptor Associated Kinase 1 (IRAK1), TNF Alpha Induced Protein 3 (TNFAIP3), Autophagy Related 5 (ATG5), Tyrosine Kinase 2 (TYK2), Signal Transducer and Activator Of Transcription 4 (STAT4), Osteopontin (OPN), Keratins (KRT), or a combination thereof. In some cases, the detecting comprises contacting the isolated nucleic acids with an additional set of probes that recognizes IFNB1, IFNE, IFNW1, ADAR, IFIT, IFI, IRF, OAS1, IRAK1, TNFAIP3, ATG5, TYK2, STAT4, OPN, KRT, or a combination thereof and the additional set of probes.

### Assays

Multiple assays may be used to process biological samples of a subject and identify/measure biomarkers or biomarker signatures. For example, a first assay may be used to process a first biological sample obtained or derived from the subject to generate a first dataset; and based at least in part on the first dataset, a second assay different from said first assay may be used to process a second biological sample obtained or derived from the subject to generate a second dataset indicative of said disease related state. The first assay may be used to screen or process biological samples of a set of subjects, while the second or subsequent assays may be used to screen or process biological samples of a smaller subset of the set of subjects. The first assay may have a low cost and/or a high sensitivity of detecting one or more disease related states (e.g., disease related complication), that is amenable to screening or processing biological samples of a relatively large set of subjects. The second assay may have a higher cost and/or a higher specificity of detecting one or more disease related states (e.g., disease related complication), that is amenable to screening or processing biological samples of a relatively small set of subjects (e.g., a subset of the subjects screened using the first assay). The second assay may generate a second dataset having a specificity (e.g., for one or more disease related states such as disease related complications) greater than the first dataset generated using the first assay. As an example, one or more biological samples may be processed using a cfRNA assay on a large set of subjects and subsequently a metabolomics assay on a smaller subset of subjects, or vice versa. The smaller subset of subjects may be selected based at least in part on the results of the first assay.

Alternatively, multiple assays may be used to simultaneously process biological samples of a subject. For example, a first assay may be used to process a first biological sample obtained or derived from the subject to generate a first dataset indicative of the disease related state; and a second assay different from the first assay may be used to process a second biological sample obtained or derived from the subject to generate a second dataset indicative of the disease related state. Any or all of the first dataset and the second dataset may then be analyzed to assess the disease related state of the subject. For example, a single diagnostic index or diagnosis score may be generated based on a combination of the first dataset and the second dataset. As another example, separate diagnostic indexes or diagnosis scores may be generated based on the first dataset and the second dataset.

The biological samples may be processed using a metabolomics assay. For example, a metabolomics assay may be used to identify a quantitative measure (e.g., indicative of a presence, absence, or relative amount) of each of a plurality of disease or condition associated metabolites in a biological sample of the subject. The metabolomics assay may be configured to process biological samples such as a blood sample or a urine sample (or derivatives thereof) of the subject. A quantitative measure (e.g., indicative of a presence, absence, or relative amount) of disease or condition associated metabolites in the biological sample may be indicative of one or more disease related states. The metabolites in the biological sample may be produced (e.g., as an end product or a byproduct) as a result of one or more metabolic pathways corresponding to disease or condition associated genes. Assaying one or more metabolites of the biological sample may comprise isolating or extracting the metabolites from the biological sample. The metabolomics assay may be used to generate datasets indicative of the quantitative measure (e.g., indicative of a presence, absence, or relative amount) of each of a plurality of disease or condition associated metabolites in the biological sample of the subject.

The metabolomics assay may analyze a variety of metabolites in the biological sample, such as small molecules, lipids, amino acids, peptides, nucleotides, hormones and other signaling molecules, cytokines, minerals and elements, polyphenols, fatty acids, dicarboxylic acids, alcohols and polyols, alkanes and alkenes, keto acids, glycolipids, carbohydrates, hydroxy acids, purines, prostanoids, catecholamines, acyl phosphates, phospholipids, cyclic amines, amino ketones, nucleosides, glycerolipids, aromatic acids, retinoids, amino alcohols, pterins, steroids, carnitines, leukotrienes, indoles, porphyrins, sugar phosphates, coenzyme A derivatives, glucuronides, ketones, sugar phosphates, inorganic ions and gases, sphingolipids, bile acids, alcohol phosphates, amino acid phosphates, aldehydes, quinones, pyrimidines, pyridoxals, tricarboxylic acids, acyl glycines, cobalamin derivatives, lipoamides, biotin, and polyamines.

The metabolomics assay may comprise, for example, one or more of: mass spectroscopy (MS), targeted MS, gas chromatography (GC), high performance liquid chromatography (HPLC), capillary electrophoresis (CE), nuclear magnetic resonance (NMR) spectroscopy, ion-mobility spectrometry, Raman spectroscopy, electrochemical assay, or immune assay.

The biological samples may be processed using a methylation-specific assay. For example, a methylation-specific assay may be used to identify a quantitative measure (e.g., indicative of a presence, absence, or relative amount) of methylation each of a plurality of disease or condition associated genomic loci in a biological sample of the subject. The methylation-specific assay may be configured to process biological samples such as a blood sample or a urine sample (or derivatives thereof) of the subject. A quantitative measure (e.g., indicative of a presence, absence, or relative amount) of methylation of disease or condition associated genomic loci in the biological sample may be indicative of one or more related states. The methylation-specific assay may be used to generate datasets indicative of the quantitative measure (e.g., indicative of a presence, absence, or relative amount) of methylation of each of a plurality of disease or condition associated genomic loci in the biological sample of the subject.

The methylation-specific assay may comprise, for example, one or more of: a methylation-aware sequencing (e.g., using bisulfite treatment), pyrosequencing, methylation-sensitive single-strand conformation analysis (MS-SSCA), high-resolution melting analysis (HRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

The biological samples may be processed using a proteomics assay. For example, a proteomics assay may be used to identify a quantitative measure (e.g., indicative of a presence, absence, or relative amount) of each of a plurality of disease or condition associated proteins or polypeptides in a biological sample of the subject. The proteomics assay may be configured to process biological samples such as a blood sample or a urine sample (or derivatives thereof) of the subject. A quantitative measure (e.g., indicative of a presence, absence, or relative amount) of disease or condition associated proteins or polypeptides in the biological sample may be indicative of one or more related states. The proteins or polypeptides in the biological sample may be produced (e.g., as an end product or a byproduct) as a result of one or more biochemical pathways corresponding to disease or condition associated genes. Assaying one or more proteins or polypeptides of the biological sample may comprise isolating or extracting the proteins or polypeptides from the biological sample. The proteomics assay may be used to generate datasets indicative of the quantitative measure (e.g., indicative of a presence, absence, or relative amount) of each of a plurality of disease or condition associated proteins or polypeptides in the biological sample of the subject.

The proteomics assay may analyze a variety of proteins or polypeptides in the biological sample, such as proteins made under different cellular conditions (e.g., development, cellular differentiation, or cell cycle). The proteomics assay may comprise, for example, one or more of: an antibody-based immunoassay, an Edman degradation assay, a mass spectrometry-based assay (e.g., matrix-assisted laser desorption/ionization (MALDI) and electrospray ionization (ESI)), a top-down proteomics assay, a bottom-up proteomics assay, a mass spectrometric immunoassay (MSIA), a stable isotope standard capture with anti-peptide antibodies (SISCAPA) assay, a fluorescence two-dimensional differential gel electrophoresis (2-D DIGE) assay, a quantitative proteomics assay, a protein microarray assay, or a reverse-phased protein microarray assay. The proteomics assay may detect post-translational modifications of proteins or polypeptides (e.g., phosphorylation, ubiquitination, methylation, acetylation, glycosylation, oxidation, and nitrosylation). The proteomics assay may identify or quantify one or more proteins or polypeptides from a database (e.g., Human Protein Atlas, PeptideAtlas, and UniProt).

### Treatment of Biological Samples

Methods described herein may comprise treatment of biological samples. In some instances, biological samples are excised or removed from a subject and treated in-vitro. In some instances, samples are removed via biopsy or non-invasive/minimally invasive sampling technique. Biological samples obtained using the methods described herein may be exposed to one or more treatments. In some instances, biological samples comprise cells, such as those obtained from biopsy. In some instances, biological samples are obtained from non-invasive or minimally-invasive techniques. In some instances, such techniques are configured to isolate specific regions or portions of a biological sample, such as a skin sample. In some instances, after exposure of biological samples to treatments, biomarker signatures are identified and compared to baseline signatures take from a subject. In some instances, such comparisons result in outcome signatures which are predictive of a subject's response to one or more treatments. Treatments in some instances are expected to reduce, minimize, treat, or cure a subject's disease or condition having a cutaneous manifestation. Biological samples are in some instances exposed to treatments for about 1, 2, 3, 4, 5, 6, 7, 8, 9, 10, 11, 12, 13, 15, 17, or about 20 days. Biological samples are in some instances exposed to treatments for at least 1, 2, 3, 4, 5, 6, 7, 8, 9, 10, 11, 12, 13, 15, 17, or at least 20 days. Biological samples are in some instances exposed to treatments for 1-20, 3-20, 5-20, 5-15, 5-10, 7-20, 7-15, 10-20, or 10-15 days. In some instances, biological samples are exposed to treatments using a titration. In some instances, a titration comprises 1×, 10×, 20×, 50×, 100×, 1000×, or 10000× increases in exposure to the one or more treatments. In some instances, biological samples are aliquoted prior to contact with one or more treatments. In some instances, each aliquot is exposed to a different treatment type or treatment level (e.g., exposure time, energy, dose, or other measurable level). In some instances, a portion of the biological sample is not exposed to a treatment (control). In some instances, prior or during contact with one or more treatments, a biological sample is analyzed for biomarker signature (e.g., treatment signature). More than one signature is in some instances obtained during exposure to treatments.

Treatments may comprise any number of methods described herein. In some instances, treatment comprises exposure to radiation. In some instances, treatment comprises phototherapy. In some instances, treatment radiation comprises ultraviolet, visible, or infrared light. In some instances, treatment comprises a therapeutic agent. In some instances, treatment the therapeutic agent is a topical or systemic agent. In some instances, treatment the therapeutic agent is a small molecule or peptide. In some instances, treatment the therapeutic agent comprises an antibody, diabody, scFv, or fragment thereof. In some instances, treatment the antibody comprises anti-TNF-a, anti-IL17A, anti-IL23p19, anti-IL-4Ralpha, or anti-IL-13. In some instances, treatment the therapeutic agent comprises a steroid or other immunosuppressant agent. In some instances, steroids include but are not limited to clobetasone, beclometasone, betamethasone, clobetasol, fluticasone and mometasone or immunosuppressant agent (e.g., cyclosporin, tacrolimus, etc.)

In some instances, treatment the therapeutic agent comprises an anti-proliferative. In some instances, a treatment is configured to reduce the expression of one or more genes overexpressed in a disease or condition. In some instances, a treatment is configured to increase the expression of one or more genes overexpressed in a disease or condition. In some instances, anti-proliferatives include but are not limited to dacarbazine (also called DTIC), temozolomide, nab-paclitaxel, paclitaxel, cisplatin, carboplatin, 5-FU (fluorouracil), aldesleukin, avelumab, cemiplimab, cobimetinib, dabrafenib, dacarbazine, imiquimod, ipilimumab, nivolumab, peginterferon alfa-2b, pembrolizumab, recombinant interferon alfa-2b, sonidegib, sylatron peginterferon alfa-2b, talimogene laherparepvec, trametinib dimethyl sulfoxide, vemurafenib, and vismodegib.

After a treatment signature is obtained, in some instances it is compared with a baseline signature. In some instances, this comparison results in an outcome signature predictive of the treatment when used on the subject, for the disease or condition.

### Machine Learning

Machine learning may be used to identify or analyze biomarker signatures, or to compare biomarker signatures (e.g., outcome signatures). The systems, methods, software, and platforms as described herein may comprise computer-implemented methods of supervised or unsupervised learning methods, including SVM, random forests, clustering algorithm (or software module), gradient boosting, logistic regression, and/or decision trees. The machine learning methods as described herein may improve generation of suggestions based on recording and analyzing any of the baseline biomarker signature, treatment biomarker signature, and outcome signature described herein. In some cases, the machine learning methods may intentionally group or separate treatment options. In some embodiments, some treatment options may be intentionally clustered or removed from any one phase of the plurality of phases of the medical care encounter.

Supervised learning algorithms may be algorithms that rely on the use of a set of labeled, paired training data examples to infer the relationship between an input data and output data. Unsupervised learning algorithms may be algorithms used to draw inferences from training data sets to output data. Unsupervised learning algorithms may comprise cluster analysis, which may be used for exploratory data analysis to find hidden patterns or groupings in process data. One example of an unsupervised learning method may comprise principal component analysis. Principal component analysis may comprise reducing the dimensionality of one or more variables. The dimensionality of a given variables may be at least 1, 5, 10, 50, 100, 200, 300, 400, 500, 600, 700, 800, 900, 1000, 1100, 1200 1300, 1400, 1500, 1600, 1700, 1800, or greater. The dimensionality of a given variables may be at most 1800, 1600, 1500, 1400, 1300, 1200, 1100, 1000, 900, 800, 700, 600, 500, 400, 300, 200, 100, 50, 10 or less.

Computer-implemented methods may comprise statistical techniques. In some embodiments, statistical techniques may comprise linear regression, classification, resampling methods, subset selection, shrinkage, dimension reduction, nonlinear models, tree-based methods, support vector machines, unsupervised learning, or any combination thereof.

A linear regression may be a method to predict a target variable by fitting the best linear relationship between a dependent and independent variable. The best fit may mean that the sum of all distances between a shape and actual observations at each point is the least. Linear regression may comprise simple linear regression and multiple linear regression. A simple linear regression may use a single independent variable to predict a dependent variable. A multiple linear regression may use more than one independent variable to predict a dependent variable by fitting a best linear relationship.

A classification may be a data mining technique that assigns categories to a collection of data in order to achieve accurate predictions and analysis. Classification techniques may comprise logistic regression and discriminant analysis. Logistic regression may be used when a dependent variable is dichotomous (binary). Logistic regression may be used to discover and describe a relationship between one dependent binary variable and one or more nominal, ordinal, interval or ratio-level independent variables. A resampling may be a method comprising drawing repeated samples from original data samples. A resampling may not involve a utilization of a generic distribution tables in order to compute approximate probability values. A resampling may generate a unique sampling distribution on a basis of an actual data. In some embodiments, a resampling may use experimental methods, rather than analytical methods, to generate a unique sampling distribution. Resampling techniques may comprise bootstrapping and cross-validation. Bootstrapping may be performed by sampling with replacement from original data and take “not chosen” data points as test cases. Cross validation may be performed by split training data into a plurality of parts.

A subset selection may identify a subset of predictors related to a response. A subset selection may comprise best-subset selection, forward stepwise selection, backward stepwise selection, hybrid method, or any combination thereof. In some instances, shrinkage fits a model involving all predictors, but estimated coefficients are shrunken towards zero relative to the least squares estimates. This shrinkage may reduce variance. A shrinkage may comprise ridge regression and a lasso. A dimension reduction may reduce a problem of estimating n+1 coefficients to a simpler problem of m+1 coefficients, where m<n. It may be attained by computing n different linear combinations, or projections, of variables. Then these n projections are used as predictors to fit a linear regression model by least squares. Dimension reduction may comprise principal component regression and partial least squares. A principal component regression may be used to derive a low dimensional set of features from a large set of variables. A principal component used in a principal component regression may capture the most variance in data using linear combinations of data in subsequently orthogonal directions. The partial least squares may be a supervised alternative to principal component regression because partial least squares may make use of a response variable in order to identify new features.

A nonlinear regression may be a form of regression analysis in which observational data are modeled by a function which is a nonlinear combination of model parameters and depends on one or more independent variables. A nonlinear regression may comprise a step function, piecewise function, spline, generalized additive model, or any combination thereof.

Tree-based methods may be used for both regression and classification problems. Regression and classification problems may involve stratifying or segmenting the predictor space into a number of simple regions. Tree-based methods may comprise bagging, boosting, random forest, or any combination thereof. Bagging may decrease a variance of prediction by generating additional data for training from the original dataset using combinations with repetitions to produce multistep of the same carnality/size as original data. Boosting may calculate an output using several different models and then average a result using a weighted average approach. A random forest algorithm may draw random bootstrap samples of a training set. Support vector machines may be classification techniques. Support vector machines may comprise finding a hyperplane that best separates two classes of points with the maximum margin. Support vector machines may constrain an optimization problem such that a margin is maximized subject to a constraint that it perfectly classifies data.

Unsupervised methods may be methods to draw inferences from datasets comprising input data without labeled responses. Unsupervised methods may comprise clustering, principal component analysis, k-Mean clustering, hierarchical clustering, or any combination thereof.

### Trained Algorithms

After using one or more assays to process one or more cell-free biological samples derived from the subject to generate one or more datasets indicative of the disease or condition, a trained algorithm may be used to process one or more of the datasets (e.g., at each of a plurality of disease or condition associated genomic loci) to determine the signatures for the disease or condition, such as baseline or treatment signatures. For example, the trained algorithm may be used to determine quantitative measures of sequences at each of the plurality of disease or condition associated genomic loci in the cell-free biological samples. The trained algorithm may be configured to identify the disease related state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a disease related state. For example, an input variable may comprise a number of sequences corresponding to or aligning to each of the plurality of disease or condition associated genomic loci. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the cell-free biological sample by the classifier. The trained algorithm may comprise a binary classifier, such that each of the one or more output values comprises one of two values (e.g., {0, 1}, {positive, negative}, or {high-risk, low-risk}) indicating a classification of the cell-free biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the cell-free biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's disease related state, and may comprise, for example, a therapeutic intervention, a duration of the therapeutic intervention, and/or a dosage of the therapeutic intervention suitable to treat a disease related condition. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof. For example, such descriptive labels may provide a prognosis of the disease related state of the subject. As another example, such descriptive labels may provide a relative assessment of the disease related state (e.g., an estimated gestational age in number of days, weeks, or months) of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the disease related state of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a disease related state (e.g., disease related complication). For example, a binary classification of samples may assign an output value of “negative” or 0 if the sample indicates that the subject has less than a 50% probability of having a disease related state (e.g., disease related complication). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a disease related state (e.g., disease related complication) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a disease related state (e.g., disease related complication) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a disease related state (e.g., disease related complication) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a disease related state (e.g., disease related complication) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a cell-free biological sample from a subject, associated datasets obtained by assaying the cell-free biological sample (as described elsewhere herein), and one or more known output values corresponding to the cell-free biological sample (e.g., a clinical diagnosis, prognosis, absence, or treatment efficacy of a disease related state of the subject). Independent training samples may comprise cell-free biological samples and associated datasets and outputs obtained or derived from a plurality of different subjects. Independent training samples may comprise cell-free biological samples and associated datasets and outputs obtained at a plurality of different time points from the same subject (e.g., on a regular basis such as weekly, biweekly, or monthly). Independent training samples may be associated with presence of the disease related state (e.g., training samples comprising cell-free biological samples and associated datasets and outputs obtained or derived from a plurality of subjects known to have the disease related state). Independent training samples may be associated with absence of the disease related state (e.g., training samples comprising cell-free biological samples and associated datasets and outputs obtained or derived from a plurality of subjects who are known to not have a previous diagnosis of the disease related state or who have received a negative test result for the disease related state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples associated with presence of the disease related state and/or cell-free biological samples associated with absence of the disease related state. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the disease related state. In some embodiments, the cell-free biological sample is independent of samples used to train the trained algorithm.

The trained algorithm may be trained with a first number of independent training samples associated with presence of the disease related state and a second number of independent training samples associated with absence of the disease related state. The first number of independent training samples associated with presence of the disease related state may be no more than the second number of independent training samples associated with absence of the disease related state. The first number of independent training samples associated with presence of the disease related state may be equal to the second number of independent training samples associated with absence of the disease related state. The first number of independent training samples associated with presence of the disease related state may be greater than the second number of independent training samples associated with absence of the disease related state.

The trained algorithm may be configured to identify the disease related state at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the disease related state by the trained algorithm may be calculated as the percentage of independent test samples (e.g., subjects known to have the disease related state or subjects with negative clinical test results for the disease related state) that are correctly identified or classified as having or not having the disease related state.

The trained algorithm may be configured to identify the disease related state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the disease related state using the trained algorithm may be calculated as the percentage of cell-free biological samples identified or classified as having the disease related state that correspond to subjects that truly have the disease related state.

The trained algorithm may be configured to identify the disease related state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the disease related state using the trained algorithm may be calculated as the percentage of cell-free biological samples identified or classified as not having the disease related state that correspond to subjects that truly do not have the disease related state.

The trained algorithm may be configured to identify the disease related state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the disease related state using the trained algorithm may be calculated as the percentage of independent test samples associated with presence of the disease related state (e.g., subjects known to have the disease related state) that are correctly identified or classified as having the disease related state.

The trained algorithm may be configured to identify the disease related state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the disease related state using the trained algorithm may be calculated as the percentage of independent test samples associated with absence of the disease related state (e.g., subjects with negative clinical test results for the disease related state) that are correctly identified or classified as not having the disease related state.

The trained algorithm may be configured to identify the disease related state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying cell-free biological samples as having or not having the disease related state.

The trained algorithm may be adjusted or tuned to improve one or more of the performance, accuracy, PPV, NPV, clinical sensitivity, clinical specificity, or AUC of identifying the disease related state. The trained algorithm may be adjusted or tuned by adjusting parameters of the trained algorithm (e.g., a set of cutoff values used to classify a cell-free biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of disease or condition associated genomic loci may be identified as most influential or most important to be included for making high-quality classifications or identifications of disease related states (or sub-types of disease related states). The plurality of disease or condition associated genomic loci or a subset thereof may be ranked based on classification metrics indicative of each genomic locus's influence or importance toward making high-quality classifications or identifications of disease related states (or sub-types of disease related states). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Computing

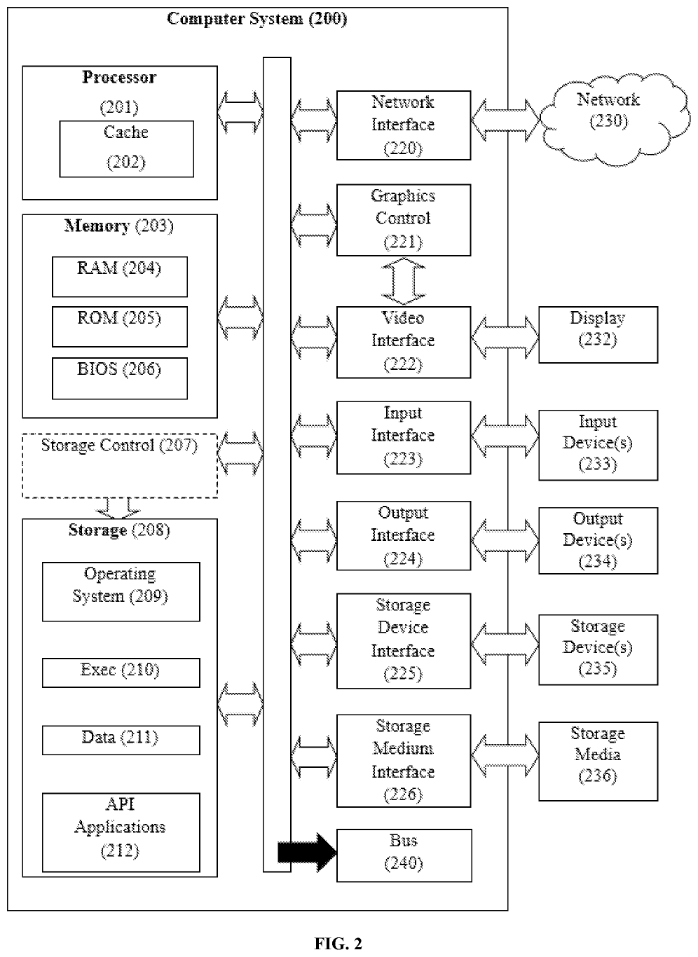

Referring to FIG. 2, a block diagram is shown depicting an exemplary machine that includes a computer system 200 (e.g., a processing or computing system) within which a set of instructions may execute for causing a device to perform or execute any one or more of the aspects and/or methodologies for static code scheduling of the methods for determining and analyzing the baseline biomarker signature, treatment biomarker signature, and outcome signature described in the present disclosure. In some embodiments, the computing system described herein generates the baseline biomarker signature, the treatment biomarker signature, or the outcome signature for predicting the therapeutic response for treating a disease or condition. The components in FIG. 2 are examples only and do not limit the scope of use or functionality of any hardware, software, embedded logic component, or a combination of two or more such components implementing particular embodiments.

Computer system 200 may include one or more processors 201, a memory 203, and a storage 208 that communicate with each other, and with other components, via a bus 240. The bus 240 may also link a display 232, one or more input devices 233 (which may, for example, include a keypad, a keyboard, a mouse, a stylus, etc.), one or more output devices 234, one or more storage devices 235, and various tangible storage media 236. All of these elements may interface directly or via one or more interfaces or adaptors to the bus 240. For instance, the various tangible storage media 236 may interface with the bus 240 via storage medium interface 226. Computer system 200 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 200 includes one or more processor(s) 201 (e.g., central processing units (CPUs) or general purpose graphics processing units (GPGPUs)) that carry out functions. Processor(s) 201 optionally contains a cache memory unit 202 for temporary local storage of instructions, data, or computer addresses. Processor(s) 201 are configured to assist in execution of computer readable instructions. Computer system 200 may provide functionality for the components depicted in FIG. 2 as a result of the processor(s) 201 executing non-transitory, processor-executable instructions embodied in one or more tangible computer-readable storage media, such as memory 203, storage 208, storage devices 235, and/or storage medium 236. The computer-readable media may store software that implements particular embodiments, and processor(s) 201 may execute the software. Memory 203 may read the software from one or more other computer-readable media (such as mass storage device(s) 235, 236) or from one or more other sources through a suitable interface, such as network interface 220. The software may cause processor(s) 201 to carry out one or more processes or one or more steps of one or more processes described or illustrated herein. Carrying out such processes or steps may include defining data structures stored in memory 203 and modifying the data structures as directed by the software.

The memory 203 may include various components (e.g., machine readable media) including, but not limited to, a random access memory component (e.g., RAM 204) (e.g., static RAM (SRAM), dynamic RAM (DRAM), ferroelectric random access memory (FRAM), phase-change random access memory (PRAM), etc.), a read-only memory component (e.g., ROM 205), and any combinations thereof. ROM 205 may act to communicate data and instructions unidirectionally to processor(s) 201, and RAM 204 may act to communicate data and instructions bidirectionally with processor(s) 201. ROM 205 and RAM 204 may include any suitable tangible computer-readable media described below. In one example, a basic input/output system 206 (BIOS), including basic routines that help to transfer information between elements within computer system 100, such as during start-up, may be stored in the memory 203.

Fixed storage 208 is connected bidirectionally to processor(s) 201, optionally through storage control unit 207. Fixed storage 208 provides additional data storage capacity and may also include any suitable tangible computer-readable media described herein. Storage 208 may be used to store operating system 209, executable(s) 210, data 211, applications 212 (application programs), and the like. Storage 208 may also include an optical disk drive, a solid-state memory device (e.g., flash-based systems), or a combination of any of the above. Information in storage 208 may, in appropriate cases, be incorporated as virtual memory in memory 203.

In one example, storage device(s) 235 may be removably interfaced with computer system 100 (e.g., via an external port connector (not shown)) via a storage device interface 225. Particularly, storage device(s) 235 and an associated machine-readable medium may provide non-volatile and/or volatile storage of machine-readable instructions, data structures, program modules, and/or other data for the computer system 200. In one example, software may reside, completely or partially, within a machine-readable medium on storage device(s) 235. In another example, software may reside, completely or partially, within processor(s) 201.

Bus 240 connects a wide variety of subsystems. Herein, reference to a bus may encompass one or more digital signal lines serving a common function, where appropriate. Bus 240 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 200 may also include an input device 233. In one example, a user of computer system 200 may enter commands and/or other information into computer system 200 via input device(s) 233. Examples of an input device(s) 233 include, but are not limited to, an alphanumeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 233 may be interfaced to bus 240 via any of a variety of input interfaces 223 (e.g., input interface 223) including, but not limited to, serial, parallel, game port, USB, FIREWIRE, THUNDERBOLT, or any combination of the above.

In particular embodiments, when computer system 200 is connected to network 230, computer system 200 may communicate with other devices, specifically mobile devices and enterprise systems, distributed computing systems, cloud storage systems, cloud computing systems, and the like, connected to network 230. Communications to and from computer system 200 may be sent through network interface 220. For example, network interface 220 may receive incoming communications (such as requests or responses from other devices) in the form of one or more packets (such as Internet Protocol (IP) packets) from network 230, and computer system 200 may store the incoming communications in memory 203 for processing. Computer system 200 may similarly store outgoing communications (such as requests or responses to other devices) in the form of one or more packets in memory 203 and communicated to network 230 from network interface 220. Processor(s) 201 may access these communication packets stored in memory 203 for processing.

Examples of the network interface 220 include, but are not limited to, a network interface card, a modem, and any combination thereof. Examples of a network 230 or network segment 230 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 230, may employ a wired and/or a wireless mode of communication. In general, any network topology may be used.

Information and data may be displayed through a display 232. Examples of a display 232 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 232 may interface to the processor(s) 201, memory 203, and fixed storage 208, as well as other devices, such as input device(s) 233, via the bus 240. The display 232 is linked to the bus 240 via a video interface 222, and transport of data between the display 232 and the bus 240 may be controlled via the graphics control 221.

In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 232, computer system 200 may include one or more other peripheral output devices 234 including, but not limited to, an audio speaker, a printer, a storage device, and any combinations thereof. Such peripheral output devices may be connected to the bus 240 via an output interface 224. Examples of an output interface 224 include, but are not limited to, a serial port, a parallel connection, a USB port, a FIREWIRE port, a THUNDERBOLT port, and any combinations thereof.

In addition or as an alternative, computer system 200 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art may appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art may also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art may recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art may recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art may also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art may also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art may also recognize that suitable video game console operating systems include, by way of non-limiting examples, Sony® PS3@, Sony® PS4@, Microsoft® Xbox 360@, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art may recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art may recognize that a web application, in various embodiments, utilizes one or more software frameworks and one or more database systems. In some embodiments, a web application is created upon a software framework such as Microsoft® NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art may also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PUP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

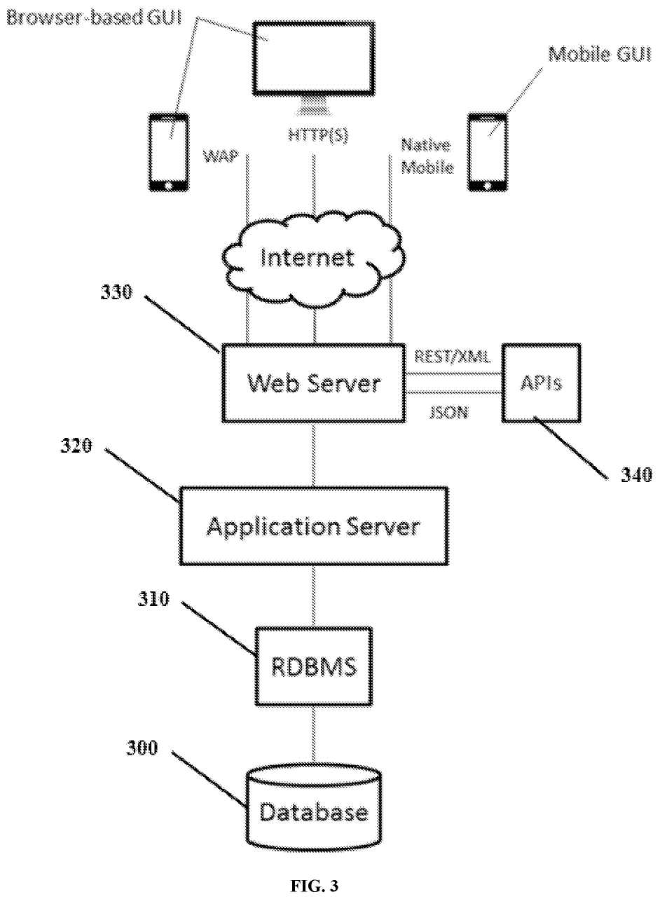

Referring to FIG. 3, in a particular embodiment, an application provision system comprises one or more databases 300 accessed by a relational database management system (RDBMS) 310. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 320 (such as Java servers, NET servers, PUP servers, and the like) and one or more web servers 330 (such as Apache, IIS, GWS and the like). The web server(s) optionally expose one or more web services via app application programming interfaces (APIs) 340. Via a network, such as the Internet, the system provides browser-based and/or mobile native user interfaces.

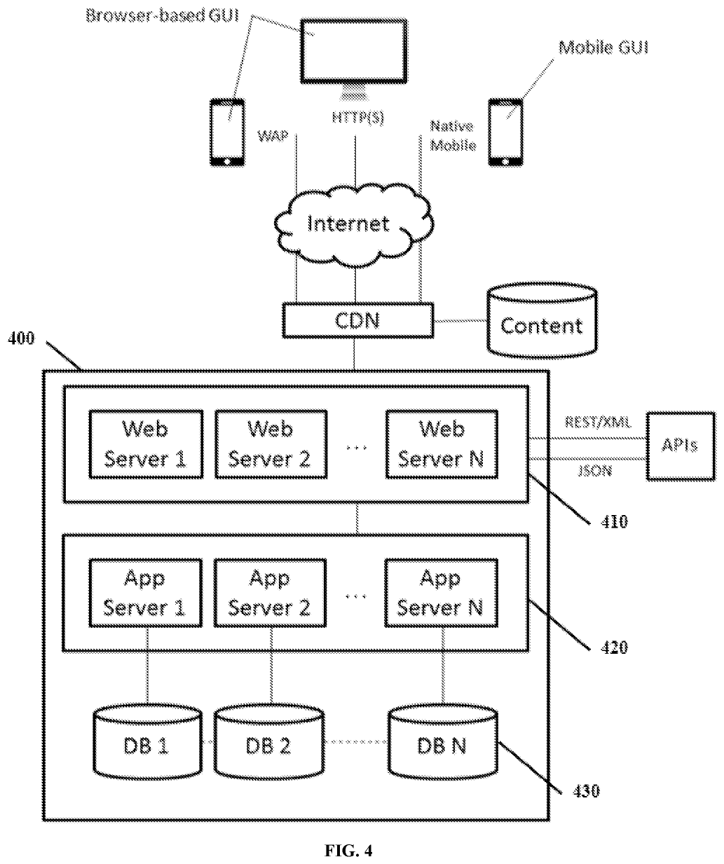

Referring to FIG. 4, in a particular embodiment, an application provision system alternatively has a distributed, cloud-based architecture 400 and comprises elastically load balanced, auto-scaling web server resources 410 and application server resources 420 as well synchronously replicated databases 430.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art may recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art may recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art may recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art may be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art may recognize that several plug-in frameworks are available that enable development of plug-ins in various programming languages, including, by way of non-limiting examples, C++, Delphi, Java™ PHP, Python™, and VB NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art may recognize that many databases are suitable for determination, storage, and retrieval of the signature information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Methods Utilizing a Computer

The methods and software described herein may utilize one or more computers. The computer may be used for determining and analyzing the baseline biomarker signature, treatment biomarker signature, and outcome signature described herein. The computer may include a monitor or other graphical interface for displaying data, results, information, or analysis of the baseline biomarker signature, treatment biomarker signature, and outcome signature described herein. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods may be transmitted over such networks or connections for reception and/or review by a party. The receiving party may be but is not limited to an individual, a health care provider or a health care manager. In one instance, a computer-readable medium includes a medium suitable for transmission of a result of an analysis of a biological sample. The medium may include a result of a subject, wherein such a result is derived using the methods described herein.

The entity obtaining the sample information may enter it into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of digital processing communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Sample Analysis Kits

Provided herein are sample analysis kits. In some instances sample analysis kits comprise components configured for obtaining a non-invasive or minimally invasive biological sample. In some instances, the analysis kit comprises an adhesive skin sample collection kit. The adhesive skin sample collection kit, in some embodiments, comprises at least one adhesive patch, a sample collector, and an instruction for use sheet. In an exemplary embodiment, the sample collector is a tri-fold skin sample collector comprising a peelable release panel comprising at least one adhesive patch, a placement area panel comprising a removable liner, and a clear panel. The tri-fold skin sample collector, in some instances, further comprises a barcode and/or an area for transcribing patient information. In some instances, the adhesive skin sample collection kit is configured to include a plurality of adhesive patches, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provide the kit operator all of the necessary information for carrying out the patch stripping method. The instructions for use sheet preferably include diagrams to illustrate the patch stripping method.

In some instances, the adhesive skin sample collection kit provides all the necessary components for performing the patch stripping method. In some embodiments, the adhesive skin sample collection kit includes a lab requisition form for providing patient information. In some instances, the kit further comprises accessory components. Accessory components include, but are not limited to, a marker, a resealable plastic bag, gloves and a cleansing reagent. The cleansing reagent includes, but is not limited to, an antiseptic such as isopropyl alcohol. In some instances, the components of the skin sample collection kit are provided in a cardboard box.

In some embodiments, the kit includes a skin collection device. In some embodiments, the skin collection device includes a non-invasive skin collection device. In some embodiments, the skin collection device includes an adhesive patch as described herein. In some embodiments, the skin collection device includes a brush. In some embodiments, the skin collection device includes a swab. In some embodiments, the skin collection device includes a probe. In some embodiments, the skin collection device includes a medical applicator. In some embodiments, the skin collection device includes a scraper. In some embodiments, the skin collection device includes an invasive skin collection device such as a needle or scalpel. In some embodiments, the skin collection device includes a needle. In some embodiments, the skin collection device includes a microneedle. In some embodiments, the skin collection device includes a hook.

Disclosed herein, in some embodiments, are kits for evaluating biomarkers in a biological sample. In some embodiments, the kit includes an adhesive patch. In some embodiments, the adhesive patch comprises an adhesive matrix configured to adhere skin sample cells from the stratum corneum of a subject. Some embodiments include a nucleic acid isolation reagent. Some embodiments include a plurality of probes that recognize at least one mutation. Disclosed herein, in some embodiments, are kits for determining a biomarkers in a skin sample, comprising: an adhesive patch comprising an adhesive matrix configured to adhere skin sample cells; a nucleic acid isolation reagent; and at least one probe that recognize at least one mutation. Disclosed herein, in some embodiments, are kits for determining a biomarker in a skin sample, comprising: an adhesive patch comprising an adhesive matrix configured to adhere skin sample cells; a sample collector, and instructions for collecting the sample and storing in the collector. In some embodiments, the kit is labeled for where the skin sample comes from on the subject (e.g., high UV exposure areas vs low UV exposure areas; or specific sampling locations such as the head (bald), temple, forehead, cheek, or nose). In some embodiments, the adhesive patch is at least 1 cm2, at least 2 cm2, at least 3 cm2, or at least 4 cm2, based on the skin sampling location.

The adhesive skin sample collection kit in some instances comprises the tri-fold skin sample collector comprising adhesive patches stored on a peelable release panel. In some instances, the tri-fold skin sample collector further comprises a placement area panel with a removable liner. In some instances, the patch stripping method involves removing an adhesive patch from the tri-fold skin sample collector peelable release panel, applying the adhesive patch to a skin sample, removing the used adhesive patch containing a skin sample and placing the applied patch on the placement area sheet. In some instances, the placement area panel is a single placement area panel sheet. In some instances, the identity of the skin sample collected is indexed to the tri-fold skin sample collector or placement area panel sheet by using a barcode or printing patient information on the collector or panel sheet. In some instances, the indexed tri-fold skin sample collector or placement sheet is sent to a diagnostic lab for processing. In some instances, the applied patch is configured to be stored on the placement panel for at least 1 week at temperatures between −80° C. and 25° C. In some embodiments, the applied patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments, the indexed tri-fold skin sample collector or placement sheet is sent to a diagnostic lab using UPS or FedEx.

### Methods of Treatment

Disclosed herein, in some embodiments, are methods of treating or determining a treatment regimen for a disease or condition having cutaneous manifestations based on the analysis of the baseline biomarker signature, treatment biomarker signature, outcome signature, or a combination thereof. In some embodiments, the treatments are recommended based on analysis of the baseline biomarker signature, treatment biomarker signature, outcome signature, or a combination thereof. In some embodiments, the treatments are recommended based on categorization of the subject's the baseline biomarker signature, treatment biomarker signature, outcome signature, or a combination thereof into one or more bins, classes, categories, qualitative actionable output, numeric actionable output, pathology score, or success rate output. In some embodiments, the baseline biomarker signature, treatment biomarker signature, outcome signature, or a combination thereof is correlated with a particular treatment which results in lowering the risk of the disease or condition in a subject. In some instances, treatment comprises administration of a treatment described herein. In some instances, a previously determined outcome signature associated with one or more treatments guides an optimum treatment for a subject. In some instances, determining optimum treatment comprises obtaining a baseline signature from a biological sample obtained from the subject, and comparing to a database of outcome signatures for a set of potential treatments. In some instances, the subject is further administered the optimum treatment.

A disease or condition described herein may have cutaneous manifestations. In some instances, disease or condition comprises a condition wherein the skin is a target or surrogate target of the cutaneous manifestation. In some instances, the disease or condition comprises an autoimmune disease, proliferative disease, or other disease having cutaneous manifestations. In some instances, the disease or condition comprises atopic dermatitis, psoriasis, allergy, Crohn's disease, lupus, asthma, or vitiligo. In some instances, the disease or condition comprises cancer or pre-cancerous conditions. In some instances, the cancer comprises melanoma or non-melanoma skin cancers. In some instances, the melanoma comprises basal cell carcinoma or squamous cell carcinoma. In some instances, the non-melanoma comprises merkel cell carcinoma or keratinosis. In some instances, the disease or condition comprises a pre-malignant condition. In some instances, the pre-malignant condition comprises actinic keratosis.

In some embodiments, the baseline biomarker signature, treatment biomarker signature, outcome signature, or a combination thereof may be used to predict therapeutic response or outcome of a treatment regimen. In some embodiments, the treatment regimen exhibits an improved therapeutic efficacy as compared with a treatment regimen not based on the analysis of the signatures described herein. The therapeutic efficacy may be determine based on the disease or condition being treated. For example, the therapeutic efficacy may be anti-proliferative effect when the disease or condition is skin cancer. In another example, the therapeutic efficacy may be modulated cytokine levels when the disease or condition is an autoimmune or an inflammatory disease. In some embodiments, the treatment regimen based on the analysis of the signatures described herein exhibits at least an increase of 0.1 fold, 0.2 fold, 0.3 fold, 0.4 fold, 0.5 fold, 0.6 fold, 0.7 fold, 0.8 fold, 0.9 fold, 1 fold, 2 fold, 5 fold, 10 fold, 20 fold, 50 fold, 100 fold, 200 fold, 500 fold, or 1000 fold in biomarkers of a disease or condition.

Some embodiments of the methods described herein comprise analyzing the baseline biomarker signature, treatment biomarker signature, outcome signature, or a combination thereof to generate an actionable output. In some embodiments, the actionable output determines the presence or severity of the disease or condition described herein. In some embodiments, the actionable output determines a treatment regimen.

### Numbered Embodiments

Provided herein are numbered embodiments 1-62. Embodiment 1. A method for preparing samples from a subject useful for predicting a response to a treatment in a subject having a disease or condition resulting in a cutaneous manifestation, comprising: a) obtaining a first biological sample and a second biological sample from the subject, b) identifying a baseline biomarker signature from the first biological sample; c) applying a treatment to the second biological in-vitro for a time period; Embodiment d) identifying a treatment signature from the second sample after the time period; and e) comparing the baseline signature with the treatment signature to determine an outcome signature to the one or more treatments. Embodiment 2. The method of embodiment 1, wherein the disease or condition is an inflammatory or autoimmune disease. Embodiment 3. The method of embodiment 1 or 2, wherein the disease or condition comprises a condition wherein the skin is a target or surrogate target of the cutaneous manifestation. Embodiment 4. The method of embodiment 2, wherein the inflammatory or autoimmune disease is atopic dermatitis, psoriasis, allergy, Crohn's disease, lupus, asthma, or vitiligo. Embodiment 5. The method of embodiment 1 or embodiment 3, wherein the disease or condition comprises cancer or pre-cancerous conditions. Embodiment 6. The method of embodiment 5, wherein the cancer is melanoma or non-melanoma skin cancers. Embodiment 7. The method of embodiment 6, wherein the melanoma comprises basal cell carcinoma or squamous cell carcinoma. Embodiment 8. The method of embodiment 6, wherein the non-melanoma comprises merkel cell carcinoma or keratinosis. Embodiment 9. The method of any one of embodiments 1-8, wherein the disease or condition comprises a pre-malignant condition. Embodiment 10. The method of embodiment 9, wherein the pre-malignant condition comprises actinic keratosis. Embodiment 11. The method of any one of embodiments 1-10, wherein the one or more treatments comprises exposure to radiation. Embodiment 12. The method of any one of embodiments 1-10, wherein the one or more treatments comprises phototherapy. Embodiment 13. The method of embodiment 11, wherein the radiation comprises ultraviolet, visible, or infrared light. Embodiment 14. The method of any one of embodiments 1-10, wherein the one or more treatments comprises a therapeutic agent. Embodiment 15. The method of any one of embodiments 1-10, wherein the therapeutic agent is a topical or systemic agent. Embodiment 16. The method of any one of embodiments 1-10, wherein the therapeutic agent is a small molecule or peptide. Embodiment 17. The method of any one of embodiments 1-10, wherein the therapeutic agent comprises an antibody, diabody, scFv, or fragment thereof. Embodiment 18. The method of embodiment 17, wherein the antibody comprises anti-TNF-a, anti-IL17A, anti-IL23p19, anti-IL-4Ralpha, or anti-IL-13. Embodiment 19. The method of embodiment 16, wherein the therapeutic agent comprises a steroid. Embodiment 20. The method of embodiment 14, wherein the therapeutic agent comprises an anti-proliferative agent. Embodiment 21. The method of any one of embodiments 1-20, wherein the first sample is non-invasively or minimally invasively sampled. Embodiment 22. The method of any one of embodiments 1-21, wherein the second sample is non-invasively or minimally invasively sampled. Embodiment 23. The method of any one of embodiments 1-21, wherein the second sample is invasively sampled. Embodiment 24. The method of any one of embodiments 1-23, wherein the first biological sample and the second biological sample are different. Embodiment 25. The method of any one of embodiments 1-24, wherein the difference between the first biological sample and the second biological sample comprises the sampling method. Embodiment 26. The method of any one of embodiments 1-25, wherein the difference between the first biological sample and the second biological sample comprises the sampling location on the subject. Embodiment 27. The method of any one of embodiments 1-26, wherein the difference between the first biological sample and the second biological sample comprises the time the sample was obtained. Embodiment 28. The method of any one of embodiments 1-27, wherein the first sample is obtained using a method comprising tape stripping, microneedles, or blood sampling. Embodiment 29. The method of any one of embodiments 1-28, wherein the first biological sample is a skin sample. Embodiment 30. The method of embodiment 29, wherein the skin sample comprises the epidermis. Embodiment 31. The method of embodiment 29, wherein the skin sample comprises the stratum corneum. Embodiment 32. The method of any one of embodiments 1-31, wherein the second biological sample is a skin sample. Embodiment 33. The method of embodiment 32, wherein the skin sample is obtained from a skin biopsy. Embodiment 34. The method of any one of embodiments 1-33, wherein the method further comprises dividing the second biological sample into a plurality of aliquots. Embodiment 35. The method of any one of embodiments 1-34, wherein the time period is up to 10 days. Embodiment 36. The method of any one of embodiments 1-34, wherein the time period is 3-15 days. Embodiment 37. The method of any one of embodiments 1-36, wherein the first biological sample and/or the second biological sample is obtained from a lesion. Embodiment 38. The method of any one of embodiments 1-37, wherein the baseline signature and the treatment signature comprise levels of at least one of a protein, lipid, mRNA, or miRNA. Embodiment 39. The method of any one of embodiments 1-37, wherein the baseline signature and the treatment signature comprise information about location and frequency of at least one genetic variant. Embodiment 40. The method of any one of embodiments 1-37, wherein the baseline signature and the treatment signature comprise information about levels of expression for one or more genes. Embodiment 41. The method of embodiment 40, wherein step e) comprises comparing weighted values of 5 or more genes. Embodiment 42. The method of embodiment 41, wherein the comparing comprises comparing weighted values of 1000 or more genes. Embodiment 43. The method of any one of embodiments 1-42, wherein the baseline signature and the treatment signature comprise information about the same set of biomarkers. Embodiment 44. The method of any one of embodiments 1-43, wherein the outcome signature comprises a predictive and/or treatment signature. Embodiment 45. The method of any one of embodiments 1-44, wherein the method further comprises measuring a second set of biomarkers obtained from the second biological sample to generate the treatment signature. Embodiment 46. A method for preparing a sample useful for differentiating a response from a non-response to a treatment in a subject with a disease having cutaneous manifestations, comprising: a) obtaining a test sample from the skin of a subject; b) identifying a baseline test biomarker signature from the test sample; c) comparing the baseline test biomarker signature with an outcome signature obtained by the method of any one of embodiments 1-45; and d) identifying whether the subject is a responder or non-responder to the treatment based on the comparison. Embodiment 47. The method of embodiment 45, wherein the test sample is obtained using a non-invasive or minimally invasive sampling method. Embodiment 48. The method of any one of embodiments 46 or 47, wherein the test sample is obtained using a method comprising tape stripping, microneedles, or blood sampling. Embodiment 49. The method of any one of embodiments 46 or 47, wherein the test sample is a skin sample. Embodiment 50. The method of embodiment 49, wherein the skin sample comprises the epidermis. Embodiment 51. The method of embodiment 49, wherein the skin sample comprises the stratum corneum. Embodiment 52. A method for preparing a samples from a subject useful for predicting a response to a treatment for a disease or condition having cutaneous manifestations comprising: a) extracting nucleic acids and/or proteins from a first biological sample of a subject, wherein the nucleic acids and/or proteins are obtained from the first biological sample; Embodiment b) excising a second biological sample from the subject; c) applying one or more treatments to the second biological sample for a time period, wherein the treatments are applied in-vitro; d) extracting nucleic acids and/or proteins from the second biological sample; e) measuring a signature for the first biological sample to generate a baseline signature; f) measuring a signature for the second biological sample to generate a treatment signature; g) comparing the baseline signature and the treatment signature to generate an outcome signature corresponding to the one or more treatments. Embodiment 53. The method of embodiment 52, wherein the skin biopsy sample is contacted with keratinocyte basal medium. Embodiment 54. The method of any one of embodiments 52-53, wherein step a) further comprises detection of nucleic acids corresponding to genes measured in the treatment signature. Embodiment 55. The method of any one of embodiments 52-54, wherein step a) further comprises detection of proteins and/or lipids measured in the treatment signature. Embodiment 56. The method of any one of embodiments 52-55, wherein the first biological sample is obtained using a non-invasive or minimally invasive sampling technique. Embodiment 57. The method of any one of embodiments 52-55, wherein the first biological sample comprises cellular material from the stratum corneum. Embodiment 58. The method of embodiment 56, wherein the stratum corneum. which has been separated from the remainder of epidermis. Embodiment 59. The method of any one of embodiments 52-58, wherein the second biological sample comprises cellular material from the epidermis. Embodiment 60. The method of any one of embodiments 52-59, wherein the second biological sample is obtained from a skin biopsy. Embodiment 61. The method of any one of embodiments 52-60, wherein comparing comprises correlating the presence or absence of one or more biomarkers from the first biological sample and the second biological sample. Embodiment 62. The method of any one of embodiments 52-60, wherein comparing comprises correlating the amount of one or more biomarkers for the first biological sample and the second biological sample.

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular.

Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease may be, for example, at least 10%, at least 20%, at least 30%, at least 40% or more, and is preferably down to a level accepted as within the range of normal for an individual without a given disease.

### EXAMPLES

The following illustrative examples are representative of embodiments of the stimulation, systems, and methods described herein and are not meant to be limiting in any way.

### Example 1. Study of the Response to Cytokine Neutralizing Antibodies in Cutaneous Biopsies In Vitro

This study aims to evaluate the effect of 5 neutralizing antibodies (anti-TNF-α, anti-IL17A, anti-IL23p19, anti-IL-4Ralpha, anti-IL-13) that block cytokine activity, their isotypes, and dexamethasone using the in vitro cultured full-thickness lesional biopsies from 10 moderate-severe psoriasis patients and 10 moderate-severe atopic dermatitis patients (Table 1). Human full-thickness cutaneous biopsies may be maintained in culture for several days without significant changes in their characteristics. This in vitro system is used to study the effect of small molecules and neutralizing antibodies on lesional samples (e.g., sampled using invasive, non-invasive or minimally invasive techniques) of different cutaneous diseases. This model allows studying the pharmacological activity of drugs on the transcriptional state of the lesions without the use of any external stimulus that may bias the information obtained. More than 500 individual explants have been performed to provide unique experience in the model to inform the current study. The nature of the translational information provided with this system is close to the results obtained in clinical trials, without the use of complex animal models that not always translates into clinic.

TABLE 1
Biopsy samples and treatment conditions

| | | Condition | Condition | Condition | Condition |
|---|---|---|---|---|---|
| Indication | Patients | 1 | 2 | 3 | 4 |
| Atopic | 1-10 | Anti- | Anti-IL-13 | Isotype | Dexamethasone |
| dermatitis | | IL4Ralpha | | | (1 μM) |
| Psoriasis | 1-10 | Anti-TNF- | Anti- | Anti- | Isotype |
| | | alpha | IL17A | IL23p19 | |

Patients. Lesional biopsies from 20 non-treated moderate-severe patients (10 psoriasis and 10 atopic dermatitis) are obtained. Skin lesions are obtained from patients of any sources through IRB approval. Two punches from lesional biopsies of 4 mm are obtained from each patient. Each punch is divided into two pieces dividing the punch longitudinally. A tape stripping is performed in the periphery of the same lesions before being biopsied. Biopsy requires some minor surgery leaving a skin wound with some suture puncture. A week after the biopsy is taken, patients are examined again, and suture puncture eliminated. All this procedure is performed by dermatologist at an operating room. Patients are without systemic treatment for 4 weeks or topical (2 weeks) before biopsies are extracted. Clinical scores of each biopsied lesion as well as time of evolution are provided for each biopsied lesion. Studies are performed under ethical committee approval of the Hospital/s providing patient's biopsies for the study.

Experimental design. Biopsies are cultured for 8 days in KBM+CaCl₂ and are changed every 2-3 days. The project may test 5 different neutralizing antibodies, isotype, and dexamethasone. Conditioned supernatants are stored at −80° for further analysis and transport. A tube of serum is also obtained from each patient for additional biomarker studies and stored at −80°. The following experimental design is proposed.

RNA. Once biopsies have been cultured for 8 days, RNA is obtained, and stored at −80° until transport for further processing and gene array analysis. Tapes obtained prior to the biopsies procedure may also be analyzed.

Tape strips. Non-invasive tape strips and the kits are used to collect skin samples from a lesion similar to the site selected for biopsies.

Analysis. Biomarker signatures from samples obtained by tape-stripping (baseline) and samples exposed to treatments (biopsied) are analyzed to generate outcome signatures which are predictive of a response to a given treatment condition.

### Example 2. Predictive Response to Treatment

A patient is suspected of having a disease or condition having cutaneous manifestations. A test sample from the patient is obtained using a non-invasive or minimally invasive technique, and a biomarker signature is identified from the sample. The biomarker signature is compared to a database of outcome signatures corresponding to specific treatments to identify a treatment for the patient. The comparison may identify the patient as a responder or non-responder to one or more treatments.

### Example 3. Patient Stratification in Clinical Study

An initial group of patients is evaluated for eligibility in a clinical study involving a treatment for a disease or condition having cutaneous manifestations. Each patient is sampled using a non-invasive or minimally invasive technique, and a biomarker signature is identified from each of the patient samples. These biomarker signatures are compared to outcome signatures obtained from in-vitro results with the treatment. Results of the comparison are used to identify patients as responders to the treatment. Patients identified as responders are selected for the clinical trial.

While the foregoing disclosure has been described in some detail for purposes of clarity and understanding, it may be clear to one skilled in the art from a reading of this disclosure that various changes in form and detail may be made without departing from the true scope of the disclosure. For example, all the techniques and apparatus described above may be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method for preparing samples from a subject useful for predicting a response to a treatment in a subject having a disease or condition resulting in a cutaneous manifestation, comprising:
a) obtaining a first biological sample and a second biological sample from the subject,
b) identifying a baseline biomarker signature from the first biological sample;
c) applying a treatment to the second biological in-vitro for a time period;
d) identifying a treatment signature from the second sample after the time period; and
e) comparing the baseline signature with the treatment signature to determine an outcome signature to the one or more treatments.

2. The method of claim 1, wherein the disease or condition is an inflammatory or autoimmune disease.

3. The method of claim 1, wherein the disease or condition comprises a condition wherein the skin is a target or surrogate target of the cutaneous manifestation.

4. The method of claim 2, wherein the inflammatory or autoimmune disease is atopic dermatitis, psoriasis, allergy, Crohn's disease, lupus, asthma, or vitiligo.

5. The method of claim 1, wherein the disease or condition comprises cancer or pre-cancerous conditions.

6. The method of claim 5, wherein the cancer is melanoma or non-melanoma skin cancers.

7. The method of claim 6, wherein the melanoma comprises basal cell carcinoma or squamous cell carcinoma.

8. The method of claim 6, wherein the non-melanoma comprises merkel cell carcinoma or keratinosis.

9. The method of claim 1, wherein the disease or condition comprises a pre-malignant condition.

10. The method of claim 9, wherein the pre-malignant condition comprises actinic keratosis.

11. The method of claim 1 wherein the one or more treatments comprises exposure to radiation.

12. The method of claim 1, wherein the one or more treatments comprises phototherapy.

13. The method of claim 11, wherein the radiation comprises ultraviolet, visible, or infrared light.

14. The method of claim 1, wherein the one or more treatments comprises a therapeutic agent.

15. The method of claim 1, wherein the therapeutic agent is a topical or systemic agent.

16. The method of claim 1, wherein the therapeutic agent is a small molecule or peptide.

17. The method of claim 1, wherein the therapeutic agent comprises an antibody, diabody, scFv, or fragment thereof.

18. The method of claim 17, wherein the antibody comprises anti-TNF-a, anti-IL17A, anti-IL23p19, anti-IL-4Ralpha, or anti-IL-13.

19. The method of claim 16, wherein the therapeutic agent comprises a steroid.

20. The method of claim 14, wherein the therapeutic agent comprises an anti-proliferative agent.

21. The method of claim 1, wherein the first sample is non-invasively or minimally invasively sampled.

22. The method of claim 1, wherein the second sample is non-invasively or minimally invasively sampled.

23. The method of claim 1, wherein the second sample is invasively sampled.

24. The method of claim 1, wherein the first biological sample and the second biological sample are different.

25. The method of claim 1, wherein the difference between the first biological sample and the second biological sample comprises the sampling method.

26. The method of claim 1, wherein the difference between the first biological sample and the second biological sample comprises the sampling location on the subject.

27. The method of claim 1, wherein the difference between the first biological sample and the second biological sample comprises the time the sample was obtained.

28. The method of claim 1, wherein the first sample is obtained using a method comprising tape stripping, microneedles, or blood sampling.

29. The method of claim 1, wherein the first biological sample is a skin sample.

30. The method of claim 29, wherein the skin sample comprises the epidermis.

31. The method of claim 29, wherein the skin sample comprises the stratum corneum.

32. The method of claim 1, wherein the second biological sample is a skin sample.

33. The method of claim 32, wherein the skin sample is obtained from a skin biopsy.

34. The method of claim 1, wherein the method further comprises dividing the second biological sample into a plurality of aliquots.

35. The method of claim 1, wherein the time period is up to 10 days.

36. The method of claim 1, wherein the time period is 3-15 days.

37. The method of claim 1, wherein the first biological sample and/or the second biological sample is obtained from a lesion.

38. The method of claim 1, wherein the baseline signature and the treatment signature comprise levels of at least one of a protein, lipid, mRNA, or miRNA.

39. The method of claim 1, wherein the baseline signature and the treatment signature comprise information about location and frequency of at least one genetic variant.

40. The method of claim 1, wherein the baseline signature and the treatment signature comprise information about levels of expression for one or more genes.

41. The method of claim 40, wherein step e) comprises comparing weighted values of 5 or more genes.

42. The method of claim 41, wherein the comparing comprises comparing weighted values of 1000 or more genes.

43. The method of claim 1, wherein the baseline signature and the treatment signature comprise information about the same set of biomarkers.

44. The method of claim 1, wherein the outcome signature comprises a predictive and/or treatment signature.

45. The method of claim 1, wherein the method further comprises measuring a second set of biomarkers obtained from the second biological sample to generate the treatment signature.

46. A method for preparing a sample useful for differentiating a response from a non-response to a treatment in a subject with a disease having cutaneous manifestations, comprising:
a) obtaining a test sample from the skin of a subject;
b) identifying a baseline test biomarker signature from the test sample;
c) comparing the baseline test biomarker signature with an outcome signature obtained by the method of claim 1; and
d) identifying whether the subject is a responder or non-responder to the treatment based on the comparison.

47. The method of claim 45, wherein the test sample is obtained using a non-invasive or minimally invasive sampling method.

48. The method of claim 46, wherein the test sample is obtained using a method comprising tape stripping, microneedles, or blood sampling.

49. The method of claim 46, wherein the test sample is a skin sample.

50. The method of claim 49, wherein the skin sample comprises the epidermis.

51. The method of claim 49, wherein the skin sample comprises the stratum corneum.

52. A method for preparing a samples from a subject useful for predicting a response to a treatment for a disease or condition having cutaneous manifestations comprising:
a) extracting nucleic acids and/or proteins from a first biological sample of a subject, wherein the nucleic acids and/or proteins are obtained from the first biological sample;
b) excising a second biological sample from the subject;
c) applying one or more treatments to the second biological sample for a time period, wherein the treatments are applied in-vitro;
d) extracting nucleic acids and/or proteins from the second biological sample;
e) measuring a signature for the first biological sample to generate a baseline signature;
f) measuring a signature for the second biological sample to generate a treatment signature;
g) comparing the baseline signature and the treatment signature to generate an outcome signature corresponding to the one or more treatments.

53. The method of claim 52, wherein the skin biopsy sample is contacted with keratinocyte basal medium.

54. The method of claim 52, wherein step a) further comprises detection of nucleic acids corresponding to genes measured in the treatment signature.

55. The method of claim 52, wherein step a) further comprises detection of proteins and/or lipids measured in the treatment signature.

56. The method of claim 52, wherein the first biological sample is obtained using a non-invasive or minimally invasive sampling technique.

57. The method of claim 52, wherein the first biological sample comprises cellular material from the stratum corneum.

58. The method of claim 56, wherein the stratum corneum is separated from the remainder of epidermis.

59. The method of claim 52, wherein the second biological sample comprises cellular material from the epidermis.

60. The method of claim 52, wherein the second biological sample is obtained from a skin biopsy.

61. The method of claim 52, wherein comparing comprises correlating the presence or absence of one or more biomarkers from the first biological sample and the second biological sample.

62. The method of claim 52, wherein comparing comprises correlating the amount of one or more biomarkers for the first biological sample and the second biological sample.
